SEQUENCE LISTING (64 sequences)

SEQ ID NO: 1 - protein, 20 aa, Artificial sequence
  artificial linker
GGGGSGGGGSGGGGSGGGGS

SEQ ID NO: 2 - protein, 5 aa, Artificial sequence
  artificial linker
GGGGS

SEQ ID NO: 3 - protein, 81 aa, Artificial sequence
  artificial antibody light chain; positions “X” are placeholders for CDR regions (see Table 4)
DIQMTQSPSSLSASVGDRVTITCWYQQKPGKAPKLLIYGVPSRFSGSGSGTDFTLTISSLQPEDFATYYCFGQGTKLTVLG

SEQ ID NO: 4 - protein, 81 aa, Artificial sequence
  artificial antibody light chain; positions “X” are placeholders for CDR regions (see Table 4)
DIQMTQSPSSLSASVGDRVTITCWYQQKPGKAPKLLIYGVPSRFSGSGSGTDFTLTISSLQPEDFATYYCFGGGTKLTVLG

SEQ ID NO: 5 - protein, 81 aa, Artificial sequence
  artificial antibody light chain; positions “X” are placeholders for CDR regions (see Table 4)
DIQMTQSPSSLSASVGDRVTITCWYQQKPGKAPKLLIYGVPSRFSGSGSGTDFTLTISSLQPEDEATYYCFGGGTKLTVLG

SEQ ID NO: 6 - protein, 81 aa, Artificial sequence
  artificial antibody light chain; positions “X” are placeholders for CDR regions (see Table 4)
DIQMTQSPSSLSASVGDRVTITCWYQQKPGKAPKLLIYGVPSRFSGSGSGTDFTLTISSLQPEDFATYYCFGTGTKVTVLG

SEQ ID NO: 7 - protein, 81 aa, Artificial sequence
  artificial antibody light chain; positions “X” are placeholders for CDR regions (see Table 4)
DIQMTQSPSSLSASVGDRVTITCWYQQKPGKAPKLLIYGVPSRFSGSGSGTDFTLTISSLQPEDEATYYCFGTGTKVTVLG

SEQ ID NO: 8 - protein, 81 aa, Artificial sequence
  artificial antibody heavy chain; positions “X” are placeholders for CDR regions (see Table 4)
EVQLVESGGGLVQPGGSLRLSCAASWVRQAPGKGLEWIGRFTISRDNSKNTVYLQMNSLRAEDTAVYYCAWGQGTLVTVSS

SEQ ID NO: 9 - protein, 11 aa, Artificial sequence
  artificial CDR library construct “X” are placeholders for amino acid residues with limited diversitys (see Table 4)
QASXNIYSNLA

SEQ ID NO: 10 - protein, 7 aa, Artificial sequence
  artificial CDR construct
RASTLAS

SEQ ID NO: 11 - protein, 12 aa, Artificial sequence
  artificial CDR library construct “X” are placeholders for amino acid residues with limited diversitys (see Table 4)
QSNYGINXYGAA

SEQ ID NO: 12 - protein, 10 aa, Artificial sequence
  artificial CDR library construct “X” are placeholders for amino acid residues with limited diversitys (see Table 4)
GFSLSSYDMX

SEQ ID NO: 13 - protein, 16 aa, Artificial sequence
  artificial CDR library construct “X” are placeholders for amino acid residues with limited diversitys (see Table 4)
IIYXSXSXYYASWAKG

SEQ ID NO: 14 - protein, 8 aa, Artificial sequence
  artificial CDR library construct “X” are placeholders for amino acid residues with limited diversitys (see Table 4)
RXNYGXDL

SEQ ID NO: 15 - protein, 11 aa, Artificial sequence
  artificial CDR construct
QASQNIYSNLA

SEQ ID NO: 16 - protein, 7 aa, Artificial sequence
  artificial CDR construct
RASTLAS

SEQ ID NO: 17 - protein, 12 aa, Artificial sequence
  artificial CDR construct
QSNYGINYYGAA

SEQ ID NO: 18 - protein, 10 aa, Artificial sequence
  artificial CDR construct
GFSLSSYDMT

SEQ ID NO: 19 - protein, 16 aa, Artificial sequence
  artificial CDR construct
IIYVSGSAYYASWAKG

SEQ ID NO: 20 - protein, 8 aa, Artificial sequence
  artificial CDR construct
RINYGLDL

SEQ ID NO: 21 - protein, 111 aa, Artificial sequence
  artificial antibody light chain
DIQMTQSPSSLSASVGDRVTITCQASQNIYSNLAWYQQKPGKAPKLLIYRASTLASGVPSRFSGSGSGTDFTLTISSLQPEDFATYYCQSNYGINYYGAAFGQGTKLTVLG

SEQ ID NO: 22 - protein, 115 aa, Artificial sequence
  artificial antibody heavy chain
EVQLVESGGGLVQPGGSLRLSCAASGFSLSSYDMTWVRQAPGKGLEWIGIIYVSGSAYYASWAKGRFTISRDNSKNTVYLQMNSLRAEDTAVYYCARINYGLDLWGQGTLVTVSS

SEQ ID NO: 23 - protein, 13 aa, Artificial sequence
  artificial CDR library construct “X” are placeholders for amino acid residues with limited diversitys (see Table 4)
QSXXXXYXNXRLS

SEQ ID NO: 24 - protein, 7 aa, Artificial sequence
  artificial CDR library construct “X” are placeholders for amino acid residues with limited diversitys (see Table 4)
XXSXLAS

SEQ ID NO: 25 - protein, 13 aa, Artificial sequence
  artificial CDR library construct “X” are placeholders for amino acid residues with limited diversitys (see Table 4)
QGEFXCXXXDCFX

SEQ ID NO: 26 - protein, 10 aa, Artificial sequence
  artificial CDR library construct “X” are placeholders for amino acid residues with limited diversitys (see Table 4)
GFPLXXXAMX

SEQ ID NO: 27 - protein, 16 aa, Artificial sequence
  artificial CDR library construct “X” are placeholders for amino acid residues with limited diversitys (see Table 4)
XIXRXXXXYYAXWXXG

SEQ ID NO: 28 - protein, 16 aa, Artificial sequence
  artificial CDR library construct “X” are placeholders for amino acid residues with limited diversitys (see Table 4)
RRXYNXXGXPXXIGDL

SEQ ID NO: 29 - protein, 13 aa, Artificial sequence
  artificial CDR library construct “X” are placeholders for amino acid residues with limited diversitys (see Table 4)
QSSXSVYXNXRLS

SEQ ID NO: 30 - protein, 7 aa, Artificial sequence
  artificial CDR library construct “X” are placeholders for amino acid residues with limited diversitys (see Table 4)
XXSXLAS

SEQ ID NO: 31 - protein, 13 aa, Artificial sequence
  artificial CDR library construct “X” are placeholders for amino acid residues with limited diversitys (see Table 4)
QGEFXCSXXDCFX

SEQ ID NO: 32 - protein, 10 aa, Artificial sequence
  artificial CDR library construct “X” are placeholders for amino acid residues with limited diversitys (see Table 4)
GFPLSXYAMX

SEQ ID NO: 33 - protein, 16 aa, Artificial sequence
  artificial CDR library construct “X” are placeholders for amino acid residues with limited diversitys (see Table 4)
MIXRXXXXYYAXWXXG

SEQ ID NO: 34 - protein, 16 aa, Artificial sequence
  artificial CDR library construct “X” are placeholders for amino acid residues with limited diversitys (see Table 4)
RRXYNXXGXPXXIGDL

SEQ ID NO: 35 - protein, 13 aa, Artificial sequence
  artificial CDR library construct “X” are placeholders for amino acid residues with limited diversitys (see Table 4)
QSSESVYNNXRLS

SEQ ID NO: 36 - protein, 7 aa, Artificial sequence
  artificial CDR library construct “X” are placeholders for amino acid residues with limited diversitys (see Table 4)
XASSLAS

SEQ ID NO: 37 - protein, 13 aa, Artificial sequence
  artificial CDR library construct “X” are placeholders for amino acid residues with limited diversitys (see Table 4)
QGEFXCSXADCFT

SEQ ID NO: 38 - protein, 10 aa, Artificial sequence
  artificial CDR construct
GFPLSSYAMI

SEQ ID NO: 39 - protein, 16 aa, Artificial sequence
  artificial CDR library construct “X” are placeholders for amino acid residues with limited diversitys (see Table 4)
MILRAGNIYYASWXKG

SEQ ID NO: 40 - protein, 16 aa, Artificial sequence
  artificial CDR library construct “X” are placeholders for amino acid residues with limited diversitys (see Table 4)
RRXYNXXGYPIGIGDL

SEQ ID NO: 41 - protein, 13 aa, Artificial sequence
  artificial CDR construct
QSSESVYNNKRLS

SEQ ID NO: 42 - protein, 7 aa, Artificial sequence
  artificial CDR construct
TASSLAS

SEQ ID NO: 43 - protein, 13 aa, Artificial sequence
  artificial CDR construct
QGEFTCSNADCFT

SEQ ID NO: 44 - protein, 10 aa, Artificial sequence
  artificial CDR construct
GFPLSSYAMI

SEQ ID NO: 45 - protein, 16 aa, Artificial sequence
  artificial CDR construct
MILRAGNIYYASWVKG

SEQ ID NO: 46 - protein, 16 aa, Artificial sequence
  artificial CDR construct
RRHYNREGYPIGIGDL

SEQ ID NO: 47 - protein, 114 aa, Artificial sequence
  artificial antibody light chain
DIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGQGTKLTVLG

SEQ ID NO: 48 - protein, 123 aa, Artificial sequence
  artificial antibody heavy chain
EVQLVESGGGLVQPGGSLRLSCAASGFPLSSYAMIWVRQAPGKGLEWIGMILRAGNIYYASWVKGRFTISRDNSKNTVYLQMNSLRAEDTAVYYCARRHYNREGYPIGIGDLWGQGTLVTVSS

SEQ ID NO: 49 - protein, 13 aa, Artificial sequence
  artificial CDR construct
QSSESVYSNNRLS

SEQ ID NO: 50 - protein, 7 aa, Artificial sequence
  artificial CDR construct
SASTLAS

SEQ ID NO: 51 - protein, 13 aa, Artificial sequence
  artificial CDR construct
QGEFSCSSVDCFS

SEQ ID NO: 52 - protein, 10 aa, Artificial sequence
  artificial CDR construct
GFPLSAYAMI

SEQ ID NO: 53 - protein, 16 aa, Artificial sequence
  artificial CDR construct
MIIRSGTVYYANWAKG

SEQ ID NO: 54 - protein, 16 aa, Artificial sequence
  artificial CDR construct
RRHYNADGYPIGIGDL

SEQ ID NO: 55 - protein, 114 aa, Artificial sequence
  artificial antibody light chain
DIQMTQSPSSLSASVGDRVTITCQSSESVYSNNRLSWYQQKPGKAPKLLIYSASTLASGVPSRFSGSGSGTDFTLTISSLQPEDFATYYCQGEFSCSSVDCFSFGQGTKLTVLG

SEQ ID NO: 56 - protein, 123 aa, Artificial sequence
  artificial antibody heavy chain
EVQLVESGGGLVQPGGSLRLSCAASGFPLSAYAMIWVRQAPGKGLEWIGMIIRSGTVYYANWAKGRFTISRDNSKNTVYLQMNSLRAEDTAVYYCARRHYNADGYPIGIGDLWGQGTLVTVSS

SEQ ID NO: 57 - protein, 13 aa, Artificial sequence
  artificial CDR construct
QSNENIYSNNRLS

SEQ ID NO: 58 - protein, 7 aa, Artificial sequence
  artificial CDR construct
SASSLAS

SEQ ID NO: 59 - protein, 13 aa, Artificial sequence
  artificial CDR construct
QGEFNCNSADCFT

SEQ ID NO: 60 - protein, 10 aa, Artificial sequence
  artificial CDR construct
GFPLNRYAML

SEQ ID NO: 61 - protein, 16 aa, Artificial sequence
  artificial CDR construct
LITRADKKYYASWAKG

SEQ ID NO: 62 - protein, 16 aa, Artificial sequence
  artificial CDR construct
RRHYNTDGYPIAIGDL

SEQ ID NO: 63 - protein, 114 aa, Artificial sequence
  artificial antibody light chain
DIQMTQSPSSLSASVGDRVTISCQSNENIYSNNRLSWYQQKPGKAPKLLIYSASSLASGVPSRFSGSGSGTDFTLTISDLEPEDFATYYCQGEFNCNSADCFTFGQGTKLTVLG

SEQ ID NO: 64 - protein, 123 aa, Artificial sequence
  artificial antibody heavy chain
EVQLVESGGGLVQPGGSLRLSCAVSGFPLNRYAMLWVRQAPGKGLEWIGLITRADKKYYASWAKGRFTISKDNSKNTVYLQMNSLRAEDTAVYYCARRHYNTDGYPIAIGDLWGQGTLVTVSS

(19) United States
(12) Patent Application Publication
(10) Pub. No.: US 2016/0368987 A1
(43) Pub. Date: Dec. 22, 2016

(54) BISPECIFIC CONSTRUCTS AND THEIR USE IN THE TREATMENT OF VARIOUS DISEASES

(71) Applicant: NUMAB AG, Wädenswil (CH)

(72) Inventors: David URECH, Jona (CH); Tea GUNDE, Zurich (CH); Sebastian MEYER, Eggenwil (CH)

(73) Assignee: NUMAB AG, Wädenswil (CH)

(21) Appl. No.: 15/031,576

(22) PCT Filed: Oct. 24, 2014
(86) PCT No.: PCT/EP2014/002876

§ 371 Date: Apr. 22, 2016

(30) Foreign Application Priority Data
Oct. 25, 2013 (EP) 13005113.9

(51) Int. Cl. C07K 16/28

(52) U.S. Cl. CPC C07K 16/2809; C07K 16/2866; C07K 2317/31; C07K 2317/622; C07K 2317/92; C07K 2317/75; C07K 2317/33; C07K 2317/94; C07K 2317/626

(57) ABSTRACT

The present invention relates to bispecific constructs that specifically bind to cytotoxic T cells and, simultaneously, to IL5R-carrying target cells, as well as nucleic acids, vectors, host cells, pharmaceutical compositions, and methods of production and use thereof, including the use of the bispecific constructs in treating inflammatory and/or autoimmune diseases and cancer.

### FIELD OF THE INVENTION

The present invention relates to bispecific constructs that specifically bind to immune effector cells and, simultaneously, to IL5R-carrying target cells, as well as nucleic acids, vectors, host cells, pharmaceutical compositions, and methods of production and use thereof, including the use of the bispecific constructs in treating diseases in which eosinophils and/or basophils are involved.

### BACKGROUND OF THE INVENTION

Interleukin (IL)-5 is a homodimeric glycoprotein that is part of the hematopoietic family of cytokines (Hamelmann et al Int Arch Allergy Immunol 1999; 120: 8-16; Weltman et al, Expert Opin Investig Drugs 2000; 9: 491-6; Gauvreau et al, Clin Exp Allergy 2009; 39: 1297-306; Kulka et al Blood 2005; 105: 592-9). IL-5 is often co-expressed with IL-3, IL-4 and GM-CSF by Th2 cells. IL-5 is also expressed by eosinophils and has been observed in the mast cells of asthmatic airways by immunohistochemistry. IL-5 expression is regulated by several transcription factors including GATA3. Interleukin-5 is involved in the differentiation, maturation, migration, development, survival, trafficking and effector function of blood and local tissue eosinophils, in addition to basophils and mast cells.

IL-5 has been associated with the cause of several allergic diseases including allergic rhinitis and asthma, wherein a large increase in the number of eosinophils in circulation, airway tissue, and induced sputum has been observed (Shen et al, J. Immunol. 170 (6): 3296-305). Given the high occurrence of eosinophils in allergic asthma, it has been widely speculated that eosinophils have an important role in the pathology of this disease (Sanderson et al, Blood 79 (12): 3101-9).

Eosinophils are terminally differentiated granulocytes found in most mammals. The principal role of these cells, in a healthy host, is the elimination of antibody-bound parasites through the release of cytotoxic granule proteins (Giembycz et al, Pharmacol. Rev. 51 (2): 213-340). Given the fact that eosinophils are the primary IL5Rα-expressing cells, it is not surprising that this cell type responds to IL-5. In fact, IL-5 is a major regulator of eosinophil accumulation in tissues, and can modulate eosinophil behavior at every stage from maturation to survival (Lopez et al, Exp. Med. 163 (5): 1085-99).

The IL-5 receptor (IL5R) is composed of an α and a βc chain. The α subunit is specific for the IL-5 molecule, whereas the βc subunit is also recognised by interleukin-3 (IL-3) and granulocyte-macrophage colony-stimulating factor (GM-CSF) (Milburn et al, Nature 1993; 363: 172-6; Dickason et al, Nature 1996; 379: 652-5; Rossjohn et al, Blood 2000; 95: 2491-8). Glycosylation of the Asn196 residue of the Rα subunit appears to be essential for binding of IL-5 and is required for the biological activities of IL-5. Both IL5Rα and βc subunits contain extracellular fibronectin-III domains, which are characteristically conserved within the class I receptor superfamily (hemopoietin receptor family) (Bazan et al, Proc Natl Acad Sci USA 1990; 87: 6934-8; Sato et al, Curr Opin Cell Biol 1994; 6: 174-9). The three fibronectin-III domains in IL5Rα form a motif that is termed “cytokine recognition motif”, which is thought to comprise certain groups of amino acid residues, which may bind either IL-5 or an antagonist (Ishino et al, Biol Chem 2004; 279: 9547-56; Ishino et al, J Biol Chem 2005; 280: 22951-61; Ishino et al, Biochemistry 2006; 45: 1106-15), suggesting the potential to tailor specific molecules for therapeutic intervention.

IL-5 and IL5R drive allergic and inflammatory immune responses characterizing numerous diseases, such as asthma, atopic dermatitis, chronic obstructive pulmonary disease, eosinophilic gastrointestinal diseases, hyper-eosinophilic syndrome, Churg-Strauss syndrome and eosinophilic nasal polyposis. Although corticosteroid therapy is the primary treatment for these diseases, a substantial number of patients exhibit incomplete responses and suffer side-effects.

Aberrant mucosal eosinophilic inflammation of the lungs is considered a hallmark of asthma and has been found to be associated with elevated levels of IL-5 in bronchial biopsies from patients with asthma (Hamid et al, J Clin Invest 1991; 87:1541-6). IL-5 overexpression in the lung epithelium of mice has been associated with increased eosinophilic inflammation, airway hyper-responsiveness (AHR) and mucus hyper-secretion (Kowal et al, Allergy Asthma Proc 2005; 26: 456-62; Coffman et al, Science 1989; 245: 308-10; Sanderson et al, Blood 1992; 79: 3101-9; Lee et al, Exp Med 1997; 185: 2143-56). IL-5 mRNA is up-regulated in the bronchial mucosa upon allergen challenge (Robinson et al, J Allergy Clin Immunol 1993; 92: 313-24), and IL-5 concentrations correlate with clinical features of asthma (Humbert et al, Am J Respir Crit Care Med 1997; 156: 704-8). Studies in mice have demonstrated a role for IL-5 in models of allergic airway inflammation. Sensitized IL-5- or IL5Ra-deficient mice are protected from mucosal eosinophilia, AHR and peribronchial fibrosis upon challenge with inhaled allergen (Foster et al, J Exp Med 1996; 183: 195-201; Tanaka et al, Am J Respir Cell Mol Biol 2004; 31: 62-8; Cho et al, Clin Investig 2004; 113: 551-60). Similar data were obtained in mice, guinea-pigs and non-human primates treated with a neutralizing anti-IL-5 mAb before allergen inhalation, whereas allergen inhalation in IL-5 transgenic mice resulted in marked accentuated eosinophilic inflammation and AHR (Tanaka et al, Am J Respir Cell Mol Biol 2004; 31:62-8; Van Oosterhout et al, Am Rev Respir Dis 1993; 147: 548-52; Akutsu et al, Immunol Lett 1995; 45: 109-16; Mauser et al, Am J Respir Crit Care Med1995; 152: 467-72). In addition, studies in mice deficient in eosinophils have identified a role for eosinophils in tissue remodelling and AHR (Lee et al, Science 2004; 305: 1773-6. Humbles et al, Science 2004; 305: 1776-9), further supporting a dominant role for IL-5 in orchestrating eosinophilia and associated consequences in models of allergic mucosal inflammation.

Although eosinophils and basophils express the IL5R on their surface, they also function as cellular sources of IL-5. CD34+ progenitor cells may also produce IL-5, and if these progenitors become an eosinophil or basophil, they may also express the IL5R. Other cellular sources of IL-5 that do not express the IL5R include Th2 cells, mast cells, invariant natural killer (NK) T cells and non-B/non-T cells (Sakuishi et al, J Immunol 2007; 179: 3452-62; Wang et al, Clin Exp Allergy 2009; 39:798-806; Fallon et al, Exp Med 2006; 203: 1105-16; Sehmi et al, J Clin Investig 1997; 100: 2466-75; Dubucquio et al, J Exp Med 1994; 179: 703-8; Ying et al, Am J Respir Cell Mol Biol 1995; 12: 477-87; Phillips et al, J Leukoc Biol 2003; 73: 165-71). Eosinophils are primarily associated with the allergic response, releasing leukotrienes such as C4, D4 and E4, as well as proteins such as eosinophil cationic protein (ECP), eosinophil-derived neurotoxin (EDN), eosinophil peroxidase and major basic protein (MBP) and numerous cytokines and chemokines such as IL-1-IL-6, IL-8, IL-10, IL-12, IL-16, GM-CSF, regulated upon activation normal T-expressed and secreted (RANTES), TGF-a, TGF-b, monocyte chemotactic protein 1 and macrophage-inflammatory protein 1a.

Basophils express a number of cytokine receptors such as IL2Ra, GM-CSFRa, IL3R, IL4R and IL5R (Valent et al, J Allergy Clin Immunol 1994; 94: 1177-83; Toba et al; Cytometry 1999; 35: 249-59), and produce IL-4 and IL-13 (Dahinden et al, Int Arch Allergy Immunol 1997; 113: 134-7). Augmented production of mediators (such as leukotrienes) in basophils has been observed with IL-5 and GM-CSF priming. Interleukin-5 has been identified as a basophilopoietin; the cytokine stimulated dose-dependent increases in histamine content of HL-60 cells and generated a greater proportion of basophil-containing, histamine-positive, eosinophil-type colonies compared to GM-CSF, IL-3 or G-CSF (Denburg et al, Blood. 1991; 77:1462-8). In contrast to its direct effect on histamine content in basophils, IL-5 does not affect histamine release or degranulation per se, but rather serves as a primer for these basophil-characteristic events, especially when combined with ATPase inhibitors such as thapsigargin (Lie et al, Clin Exp Allergy 2000; 30: 882-90; Bischoff et al, J Exp Med 1990; 172: 1577-82). IL-5 has been shown to amplify allergen induced histamine release from basophils from patients with allergic rhinitis; this effect was comparatively greater with IL-3 than with IL-5. Similarly, a greater expression of IL-3R than either IL5R or GM-CSFR has been found on basophils, compared with eosinophils, which expressed the highest level of IL5R (Koval et al, Allergy Asthma Proc 2005; 26: 456-62).

What is known is that in good health, eosinophils—following an eotaxin-1 gradient—migrate predominantly to the gastrointestinal tract and to lesser degrees to the thymus, spleen, lymph nodes and the uterus (Kato et al, Anat Rec 1998; 252: 418-25). In disease states, eosinophils may migrate to a variety of organs such as the nose, lung, oesophagus, heart and skin among others. Despite prevalent thinking that eosinophil progenitor maturation and differentiation occur primarily in the bone marrow, there is considerable emerging evidence that differentiation of eosinophils also occurs within tissues having an allergic response at the airway level, such as the airways in atopic asthma (Rosenberg et al, J Allergy Clin Immunol 2007; 119: 1303-10).

Two monoclonal antibodies have been designed to neutralize IL-5 (mepolizumab (Robinson, Expert Rev Respir Med. 2013; 7:13-7) and reslizumab (Walsh, Curr Opin Mol Ther. 2009; 11: 329-36)). Both antibodies have demonstrated the ability to reduce blood and tissue eosinophil counts. One additional monoclonal antibody, benralizumab (MEDI-563; Ghazi et al, Expert Opin Biol Ther. 2012; 12: 113-8), has been developed to target IL5R and attenuate eosinophilia through antibody-dependent cellular cytotoxicity. All three monoclonal antibodies are being clinically evaluated. Antisense oligonucleotide technology targeting the common βc IL5R subunit is also being used therapeutically to inhibit IL-5-mediated effects (TPI ASM8). Small interfering RNA technology has also been used therapeutically to inhibit the expression of IL-5 in animal models. Such targeting has focused most commonly on asthma as well as on Churg-Strauss syndrome due to strong eosinophilic inflammation in affected tissues, and has also been used to treat eosinophil-associated gastrointestinal disorders, hyper-eosinophilic syndrome (HES), atopic dermatitis (AD) and idiopathic pulmonary fibrosis.

Although mAb-targeted inhibition of IL-5 itself (e.g. with mepolizumab and reslizumab) has been effective in reducing eosinophil counts in several disease states, the clinical responses have been consistently suboptimal and are not entirely understood.

The two IL-5 blocking antibodies reslizumab and mepolizumab as well as the IL5R targeting antibody benralizumab are administered systemically by parenteral injection for the therapy of asthma. All three molecules showed near complete elimination of eosinophils in bone marrow and in the blood. However, none of them was able to completely eliminate eosinophils in the lung tissue or the sputum. It may, however, be critical to target eosinophils and basophils at the local site because eosinophil precursors can differentiate in the lung, so that systemic blockade of IL-5 signaling may not prevent eosinophil differentiation, particularly as IL-5 is mainly produced by local T helper cells. Further, lung resident eosinophils have been described to be sufficient for eliciting asthma exacerbations. In line with this, also other clinical responses (e.g. reduction of exacerbation frequency and hospitalization rates) were suboptimal with systemically administered antibodies.

There are several possible explanations for the incomplete depletion of local eosinophils upon systemic administration of IL-5 blocking antibodies. First, there is the poor local availability of full-length IgG in the lung after systemic administration, since the availability in the lung of pulmonary drugs that are delivered systemically is generally low with only 2-5% of the delivered dose in the target organ, even with small molecule drugs (Kane et al., Drug Targets, 2013; 12: 81-87). Second, although eosinophils generally require the IL-5 signal for differentiation, proliferation and survival, there are other factors, such as eotaxin that have at least in part redundant functions, so that even if local concentration of IL-5 blockers would be sufficient to completely neutralize IL-5, this may not completely inhibit eosinophil differentiation and proliferation (Foster et al., J Exp Med. 1996; 183: 195-201; Nishinakamura et al., Blood. 1996; 88: 2458-2464; Foster et al., Immunol. Rev. 2001; 179: 173-181). Third, certain lung resident eosinophils are completely independent of IL-5 signaling (Foster et al., J Exp Med. 1996; 183; 195-201; Hogan et al., J Immunol. 1998; 161: 1501-1509; Foster et al., TRENDS in Molecular Medicine, 2002; 8: 162-167).

Benralizumab (formerly MEDI-563) is a humanized anti-IL5Ra mAb that binds with comparably weak affinity to the alpha chain of the IL5R (K_{D}˜1 nM) to block IL-5 function and induce apoptosis of eosinophils and basophils through antibody-dependent cell-mediated cytotoxicity. This may provide a more promising mechanism of action as it directly eliminates eosinophils irrespective of their possibly varying dependence from different cytokines. However, similar to the IL-5 blocking antibodies mepolizumab and reslizumab, also benralizumab is administered systemically by parenteral injection. Thus, local availability in the lung is limited by the poor tissue penetration capability of full-size IgGs, which may be the reason for the incomplete elimination of lung eosinophils in clinical studies. Furthermore, IL5R expression levels in eosinophils may vary, and it could be that the comparably weak affinity of benralizumab to IL5R (K_{D}˜nM) in combination with the similarly weak affinity to CD16 on NK cells (K_{D}˜45 nM) is insufficient to induce lysis also in eosinophils with low IL5R expression.

In order to increase local concentrations of the abovementioned biotherapeutics, topical administration, such as inhalation, presents a highly attractive route of administration. However, despite the success of biologic drugs as systemic therapies, only few protein drugs (e.g. Exubera, Pulmozyme), but no antibodies, or antibody-based drugs, have been approved for delivery via inhalation so far. A monoclonal anti-IgE antibody delivered via nebulization had been tested clinically as a treatment for asthma. However, in contrast to systemic delivery of anti-IgE, the inhaled antibody was not effective (Fahy et al. Am J Respir Crit Care Med. 1990; 160: 1023-1027). The authors of this study suggest that the nebulized full-size antibody may not be capable of crossing the epithelial barrier to access the vascular space and interstitial tissue to neutralize local IgE. An alternative explanation for the failure would be that the antibody did not retain its activity following nebulization, as antibodies frequently aggregate upon the stress of nebulization (Mailet et al, Pharm res, 2008; 25: 1318-1326).

A promising approach for the antibody-based treatment of various cancer diseases is the redirection of immune effector cells to specifically lyse target cells using bispecific antibodies. The bispecific antibodies recognize a particular antigen on the surface of a target cell and, simultaneously, an activating surface molecule of an immune effector cell, such as a natural killer (NK) cell or a cytotoxic T (Tc) cell, to thereby kill the target cells.

The bispecific antibody concept is, for example, used in cancer therapy where bispecific antibodies are employed that bind to a cancer antigen on cancer cells and, simultaneously, to the epsilon chain of CD3 presented on, for example, cytotoxic T cells. A well-known example of such a bispecific antibody construct is “blinatumomab”, an antibody in the BiTE (bi-specific T cell engager) format, for the treatment of non-Hodgkin's lymphoma and acute lymphoblastic leukemia (Nagorsen et al, Pharmacol Ther. 2012; 136: 334-42). Blinatumomab was developed by Micromet and simultaneously binds to the cancer antigen CD19 as well as to CD3 on the surface of cytotoxic T cells, thereby linking these two cell types together and activating the cytotoxic T cell to lyse the target cancer cell

There is an unmet need for potent therapeutics specifically targeting eosinophils and basophils, suitable for topical application and with a molecular weight that allows for efficient tissue penetration. In particular, the molecules for use in such treatments are required that are stable, easy to produce, highly specific for a given target antigen, and have a low immunogenicity.

The solution for this problem that has been provided by the present invention, i.e. bispecific constructs with high affinity for IL5R obtained by genetic immunization of rabbits and screening of affinity matured memory B-cells, has so far not been achieved or suggested by the prior art.

### SUMMARY OF THE INVENTION

The present invention relates to bispecific constructs that specifically bind to immune effector cells and, simultaneously, to IL5R-carrying target cells, wherein the bispecific constructs can be administered topically.

Thus, in a first aspect, the present invention relates to a bispecific construct comprising at least one first binding moiety and at least one second binding moiety, wherein said first binding moiety specifically binds to a first antigen present on a cytotoxic effector T (Tc) cell, and said second binding moiety specifically binds to the IL-5 receptor (IL5R) present on the surface of a target cell, particularly wherein the target cell is an eosinophil or basophil cell, particularly wherein the second binding moiety has a dissociation constant K_{D} of 10⁻¹⁰ M or less, particularly of less than 5×10-11 M.

In a second aspect, the present invention relates to a nucleic acid or nucleic acids encoding the bispecific construct according to the present invention.

In a third aspect, the present invention relates to a vector or vectors comprising the nucleic acid or nucleic acids according to the present invention.

In a fourth aspect, the present invention relates to a host cell or host cells comprising the vector or vectors according to the present invention.

In a fifth aspect, the present invention relates to a method for producing the bispecific construct according to the present invention, comprising (i) providing a nucleic acid or nucleic acids according to the present invention, or a vector or vectors according to the present invention, expressing said nucleic acid or nucleic acids or said vector or vectors and collecting said bispecific construct from the expression system, or (ii) providing a host cell or host cells according to the present invention, culturing said host cell or said host cells; and collecting said bispecific construct from the cell culture.

In a sixth aspect, the present invention relates to a pharmaceutical composition comprising the bispecific construct according to the present invention and a pharmaceutically acceptable carrier.

In a seventh aspect, the present invention relates to a bispecific construct according to the present invention, or the pharmaceutical composition according to the present invention, for use in the treatment of an indication in which eosinophils and/or basophils are critically involved in, particularly an allergic or inflammatory disease, particularly an allergic or inflammatory disease selected from asthma, atopic dermatitis, chronic obstructive pulmonary disease, eosinophilic gastrointestinal diseases, hyper-eosinophilic syndrome, Churg-Strauss syndrome, and eosinophilic nasal polyposis.

In an eighth aspect the present invention relates to bispecific antibody fragments binding to CD3 and to IL5R that have a midpoint of thermal unfolding of at least 55° C., particularly more than 60° C., more particularly more than 65° C., and most particularly more than 70° C.

In an ninth aspect the present invention relates to bispecific antibody fragments binding to CD3 and to IL5R that show less than 20%, particularly less than 15%, more particularly less than 12%, even more particularly less than 10 and most particularly less than 5% loss in. the monomer content of the single-chain diabody (scDb) when incubated at concentrations of 10 mg/ml in a simple saline buffer at 37° C. for 28 days.

In a tenth aspect, the present invention relates to a kit comprising (i) a bispecific construct or a pharmaceutical composition according to the present invention, and (ii) one or more of (x) a nebulizer; (y) a buffer or solvent for preparing a suspension or solution of the bispecific construct according to (i) to be nebulized; and/or (z) one or more ancillary reagents and/or tools for preparing a suspension or solution of the bispecific construct according to (i) to be nebulized.

### FIGURES

FIG. 1 shows binding of anti-CD3×anti-IL5R scDbs to Jurkat T-cells and CHO-IL5R cells. Binding of A) Construct 1, B) Construct 2 and C) Construct 3 to Jurkat T-cells and CD3-negative Jurkat cells and binding of D) Construct 1, E) Construct 2 and F) Construct 3 to IL5R-CHO cells as well as wild-type CHO cells was assessed by flow cytometry. Construct 1, Construct 2 and Construct 3 have the same anti-IL5R moiety but 3 different anti-CD3 moieties that bind to CD3 with diverse affinities (1.15×10-8 M for Construct 1, 2.96×10-8 M for Construct 2, and 1.23×10-7 M for Construct 3).

FIG. 2 shows the specific stimulation of interleukin-2 secretion by cross-linking of cytotoxic T-cells with target cells by scDbs. CD8+ T-cells were incubated with increasing concentrations of scDbs in presence of CHO-IL5R or CHO cells. Interleukin-2 concentrations in culture supernatants were measured by ELISA after 16 hours of incubation.

FIG. 3 shows the specific lysis of human IL5R-expressing CHO cells by anti-CD3×anti-IL5R scDbs. CD8+ T-cells were incubated with increasing concentrations of scDbs in presence of CHO-IL5R or CHO cells. Target cells (CHO-IL5R and CHO) were labeled with cell tox green dye and cell lysis was determined by measurement of fluorescence intensity after 88 hours of incubation.

FIG. 4 shows normalized SE-HPLC chromatograms of Construct 1, Construct 2, and Construct 3 at concentrations of 1 and 10 g/l at day 0 (grey) and after incubation at 37° C. for 28 days (black). The main peak corresponds to the scDb monomer (*) in addition to the matrix peak at higher retention time some samples show a minor dimer peak or shoulder.

FIG. 5 shows the time-resolved loss of monomer content observed for Construct 1, Construct 2, and Construct 3 at concentrations of 1 and 10 g/l at 37° C.

### DETAILED DESCRIPTION

The present invention provides a bi-specific antibody fragment for the topical therapy of allergic diseases and other indications with critical involvement of eosinophils/basophils. The bispecific antibody fragment binding to IL5R on eosinophils/basophils and to an antigen, for example, CD3ε on T cells induces targeted lysis of eosinophils by cytotoxic T cells. The low molecular weight of the antibody fragment allows for efficient penetration into the lung tissue. Further the outstanding stability of the molecule supports its administration by inhalation of a nebulized formulation.

Thus, in a first aspect, the present invention relates to a bispecific construct comprising at least one first binding moiety and at least one second binding moiety, wherein said first binding moiety specifically binds to a first antigen present on a cytotoxic effector T (Tc) cell, and said second binding moiety specifically binds to the IL-5 receptor (IL5R) present on the surface of a target cell, particularly wherein the target cell is an eosinophil or basophil cell, particularly wherein the second binding moiety has a dissociation constant K_{D} of 10⁻¹⁰ M or less, particularly of less than 10⁻¹¹M.

Within the meaning of the present invention, the term “construct” refers to any chemical entity so long as it exhibits the desired binding activity. Thus, the term “construct” is used in the broadest sense and specifically covers protein-based molecules, including recombinant antibodies and fragments thereof comprising one or more antibody-based domains or binding fragments thereof. Specific examples include, but are not limited to, monoclonal chimeric antibodies, humanized antibodies, single-chain diabodies and the like. Furthermore, the term “comprise” as used within the present invention, for example in conjunction with the term “construct”, encompasses both “includes” and “consists of”.

The term “bispecific”, as used herein, is intended to refer to a construct having two different antigen specificities. This means that a bispecific construct is capable of simultaneously binding to at least one antigen “A” and at least one antigen “B”, wherein A and B are not the same. Thus, whilst having two different antigen specificities, a bispecific construct of the present invention does not necessarily have only two binding moieties, one for each targeted antigen, but may also include more than two binding moieties. Furthermore, the term “antigen”, as used herein, is to be interpreted in a broad sense and includes any target moiety that is bound by the binding moieties of the bispecific construct of the present invention.

As used in the present invention, the terms “specific” or “specifically” are intended to mean that the first and second binding moieties are able to discriminate between their respective target molecules (i.e. between the first and second antigen) and/or one or more reference molecule(s). Thus, in its broadest sense, “specific binding” or “specifically binding” refers to the first and second binding moieties' ability to discriminate between the first antigen on the surface of a Tc cell and the second antigen on the surface of a target cell and/or between other target molecules that are related to or not related to the first antigen and/or the second antigen (i.e., IL5R).

The binding specificity of a specific binding moiety can be determined as known in the art using, for example, surface plasma resonance (SPR), western blot, enzyme-linked immunosorbent assay (ELISA), radioimmunoassay (RIA or IRMA), enhanced chemiluminescence (ECL), and peptide scan analysis.

If an ELISA assay is used, the scoring can be carried out by means of a standard color development reaction, for example by using horseradish peroxidase (HRP)-conjugated second antibodies in a HRP, H202, tetramethyl benzidine system. The optical density of the color development in the reaction vessel (e.g. well) at a given wavelength is a measure of the binding specificity. A typical background signal (negative reaction) may be about 0.1 OD, whereas a typical signal for a positive reaction may be about 1.0 OD or higher, resulting in a signal to noise ratio of 10:1 or higher. Typically, the determination of the binding specificity is carried out using a set of about three to five unrelated biomolecules, such as milk powder, BSA, transferrin and the like, rather than using only a single reference biomolecule.

In accordance with the present invention, the bispecific construct is particularly designed in such a way that the killing of IL5R-expressing target cells by Tc cells is highly efficient. Such efficient killing generally involves the ability of the bispecific construct to effectively redirect Tc cells to lyse IL5R-expressing target cells. The term “efficient”, as used herein, means that the bispecific construct of the present invention typically shows an in vitro EC₅₀ (determined as described in the Examples) ranging from 10 to 10,000 pg/ml, particularly from 3,000 to 7,000 pg/ml, particularly from 5,000 to 6,000 pg/ml, and is able to induce redirected lysis of about 50% of the target cells through Tc cells at a ratio of Tc cells to target cells of from 1:1 to 50:1, particularly from 10:1 to 25:1, more particularly from 2:1 to 10:1. As used herein above and below, the terms “about” and “approximately” refer to ±10% of the indicated value or range.

Furthermore, the bispecific construct of the present invention is particularly capable of cross-linking a stimulated as well as an (otherwise) unstimulated Tc cell and the target cell in such a way that the target cell is lysed. This offers the advantage that no generation of target-specific T cell clones or common antigen presentation by dendritic cells is required for the bispecific construct to exert its desired activity. In fact, the bispecific construct of the present invention is particularly capable of redirecting Tc cells to lyse the target cells in the absence of other activating signals. More particularly, if the first binding moiety of the bispecific construct specifically binds to CD3, particularly to CD3E, signaling through CD28 and/or IL-2 is not required for redirecting Tc cells to lyse the target cells. The high potential to activate non-target specific and/or unstimulated Tc cells is considered to be an important feature of the bispecific construct of the present invention and is believed to contribute to the efficient killing of target cells.

In particular embodiments, said first binding moiety specifically binds to an antigen selected from CD3 and CD28.

In a particular embodiment, said first binding moiety specifically binds to CD3, particularly to the epsilon chain of CD3 (CD3E), more particularly to an agonistic epitope of CD3E.

In a particular embodiment of the present invention, the first binding moiety binds specifically to CD3, more particularly to the epsilon chain of CD3 (CD3E), and most particularly to an agonistic epitope of CD3E. The term “agonistic epitope”, as used herein, means (a) an epitope that, upon binding of the bispecific construct of the present invention, optionally upon binding of several bispecific constructs on the same cell, allows said bispecific constructs to activate TCR signaling and induce T cell activation, and/or (b) an epitope that is solely composed of amino acid residues of the epsilon chain of CD3 and is accessible for binding by the bispecific construct of the present invention, when presented in its natural context on Tc cells (i.e. surrounded by the TCR, the CD3γ chain, etc.), and/or (c) an epitope that, upon binding of the bispecific construct of the present invention, does not lead to stabilization of the spatial position of CD3E relative to CD3γ.

In another particular embodiment of the present invention, instead of binding to Tc cells, the first binding moiety specifically binds to a component of the complement system, such as C1q. C1q is a subunit of the C1 enzyme complex that activates the serum complement system.

In an alternative embodiment, the present invention also contemplates the use of a first binding moiety that specifically binds to an Fc receptor, in particular to an Fc gamma receptor (FcγR). The FcγR may be an FcγRIII present on the surface of natural killer (NK) cells or one of FcγRI, FcγRIIA, FcγRIIB1, FcγRIIB2, and FcγRIIIB present on the surface of macrophages, monocytes, neutrophils and/or dendritic cells.

In such embodiment, the first binding moiety particularly is an Fc region or functional fragment thereof. In the present context, a “functional fragment” refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. Particularly, a functional Fc fragment is capable of competitively inhibiting the binding of the original, full-length Fc portion to an FcR such as the activating FcγRI. Particularly, a functional Fc fragment retains at least 30%, 40%, 50%, 60%, 70%, 80%, 90% or 95% of its affinity to an activating FcγR.

Within such embodiment of the present invention, the Fc region or functional fragment thereof is particularly an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g. FcγRIIIA (CD16A) expressed on natural killer (NK) cells) and/or a decreased binding to an inhibitory receptor (e.g. FcγRIIB1/B2 (CD32B)).

Suitable alterations within the present invention include altering glycosylation patterns, in particular afucosylation (also referred to as “defucosylation”), mutations (point mutations, deletions, insertions) and fusions with oligo- or polypeptides. Known techniques for altering glycosylation patterns include overexpression of heterologous β1,4-N-acetylglucosaminyltransferase III in the antibody-producing cell (known as the Glycart-Roche technology) and knocking out of the gene encoding α-1,6-fucosyltransferase (FUT8) in the antibody-producing cell (the Potelligent technology from Kyowa Hakko Kirin). Specific examples of enhancing mutations in the Fc part include those described in Shields et al., J. Biol. Chem. 276:6591-6604 (2001), which is incorporated herein in its entirety.

In particular embodiments, said construct allows for efficient killing of said target cell by the Tc cell and/or wherein said Tc cell is a stimulated or an unstimulated Tc cell.

In particular embodiments, said first and second binding moieties are arranged relative to each other in such a manner that the part of the first binding moiety recognizing the first antigen and the part of the second binding moiety recognizing IL5R project, relative to the center of the bispecific construct, outward in essentially opposite directions.

The first and second binding moieties are not structurally limited so long as they specifically bind to the desired first and second antigens. However, the first and second binding moieties generally consist of or are formed of one or more oligo- or polypeptides or parts thereof. Particularly, the first and second binding moieties are antibody-based binding moieties, which typically comprise at least one antibody variable domain or binding fragment thereof.

In particular embodiments of the present invention, the first binding moiety and/or the second binding moiety is an antibody-based binding moiety, particularly an antibody-based binding moiety comprising a heavy chain variable domain (VH) or binding fragment thereof, more particularly an antibody-based binding moiety comprising a heavy chain variable domain (VH) or binding fragment thereof and a light chain variable domain (VL) or binding fragment thereof. The term “binding fragment”, as used herein, refers to a portion of a given domain, region or part, which is (either alone or in combination with another domain, region or part thereof) still functional, i.e. capable of binding to the first or second antigen recognized by the bispecific construct.

In particular embodiments, the bispecific construct is an antibody format selected from the group consisting of a single-chain diabody (scDb), a tandem scDb (Tandab), a linear dimeric scDb (LD-scDb), a circular dimeric scDb (CD-scDb), a bispecific T-cell engager (BiTE; tandem di-scFv), a disulfide-stabilized Fv fragment (Brinkmann et al., Proc Natl Acad Sci USA. 1993; 90: 7538-7542), a tandem tri-scFv, a tri(a)body, bispecific Fab2, di-miniantibody, tetrabody, scFv-Fc-scFv fusion, di-diabody, DVD-Ig, IgG-scFab, scFab-dsscFv, Fv2-Fc, IgG-scFv fusions, such as bsAb (scFv linked to C-terminus of light chain), BslAb (scFv linked to N-terminus of light chain), Bs2Ab (scFv linked to N-terminus of heavy chain), Bs3Ab (scFv linked to C-terminus of heavy chain), TslAb (scFv linked to N-terminus of both heavy chain and light chain), Ts2Ab (dsscFv linked to C-terminus of heavy chain), and Knob-into-Holes (KiHs) (bispecific IgGs prepared by the KiH technology) and DuoBodies (bispecific IgGs prepared by the Duobody technology), a VH and a VL domain, each fused to one C-terminus of the two different heavy chains of a KiHs or DuoBody such that one functional Fv domain is formed, Particularly suitable for use herein is a single-chain diabody (scDb), in particular a bispecific monomeric scDb. For reviews discussing and presenting various bispecific constructs see, for example, Chan Carter, Nature Reviews Immunology 10 (2010) 301-316; Schubert et al., Antibodies 1 (2012) 2-18; Byrne et al., Trends in Biotechnology 31 (2013) 621; Metz et al., Protein Engineering Design & Selection. 2012; 25:571-580).

In a particular embodiment of the present invention, the VH domain of the first and second antibody-based binding moieties of the bispecific construct comprises rabbit heavy chain complementarity determining regions (CDRs) grafted onto human heavy chain framework (FW) regions, and the VL domain of the first and second antibody-based binding moieties of the bispecific construct comprises rabbit light chain CDRs grafted onto human light chain FW regions.

The heavy chain and light chain CDRs of the first antibody-based binding moiety are particularly derived from a rabbit antibody obtained by immunization of a rabbit with the full-length epsilon chain of human CD3 the full-length, CD28 or the full-length C1q. The immunization with the full-length chain of CD3E, CD28 or C1q is suitably conducted by DNA immunization of a rabbit with a plasmid encoding the full-length chain of human CD3E, CD28 or C1q, or, alternatively, with the purified extracellular domain of the epsilon chain of CD3, or with the purified extracellular chain of CD28, or with the purified C1q. Further, the heavy chain and light chain CDRs of the second antibody-based binding moiety are particularly derived from a rabbit antibody obtained by immunization of a rabbit either with the purified extracellular domain of IL5R or with a plasmid expressing the full-length IL5R.

The bispecific constructs of the present invention can be produced using any convenient antibody manufacturing method known in the art (see, e.g., Fischer, N. & Leger, O., Pathobiology 74:3-14 (2007) with regard to the production of bispecific constructs; and Hornig, N. & Farber-Schwarz, A., Methods Mol. Biol. 907:713-727, 2012 with regard to bispecific diabodies and tandem scFvs). Specific examples of suitable methods for the preparation of the bispecific construct of the present invention further include, inter alia, the Genmab (Labrijn et al., Proc Natl Acad Sci USA. 2013 Mar. 26; 110(13):5145-50) and Merus (de Kruif et al., Biotechnol Bioeng. 2010 Aug. 1; 106(5):741-50) technologies. Methods for production of bispecific antibodies comprising a functional antibody Fc part are also known in the art (see, e.g., Zhu et al., Cancer Lett. 86:127-134 (1994)); Suresh et al., Methods Enzymol. 121:210-228 (1986)).

These methods typically involve the generation of monoclonal antibodies, for example by means of fusing myeloma cells with the spleen cells from a mouse that has been immunized with the desired antigen using the hybridoma technology (see, e.g., Yokoyama et al., Curr. Protoc. Immunol. Chapter 2, Unit 2.5, 2006) or by means of recombinant antibody engineering (repertoire cloning or phage display/yeast display) (see, e.g., Chames & Baty, FEMS Microbiol. Letters 189:1-8 (2000)), and the combination of the antigen-binding domains or fragments or parts thereof of two different monoclonal antibodies to give a bispecific construct using known molecular cloning techniques.

The bispecific constructs of the present invention are particularly humanized in order to reduce immunogenicity and/or to improve stability. Techniques for humanization of antibodies are well-known in the art. For example, one technique is based on the grafting of complementarity determining regions (CDRs) of a xenogeneic antibody onto the variable light chain VL and variable heavy chain VH of a human acceptor framework (see, e.g., Jones et al., Nature 321:522-525 (1986); and Verhoeyen et al., Science 239:1534-1536 (1988)). In another technique, the framework of a xenogeneic antibody is mutated towards a human framework. In both cases, the retention of the functionality of the antigen-binding portions is essential (Kabat et al., J. Immunol. 147:1709-1719 (1991)).

In particular embodiments, said bispecific scDb comprises two variable heavy chain domains (VH) or fragments thereof and two variable light chain domains (VL) or fragments thereof connected by linkers L1, L2 and L3 in the order V_{H}A-L1-V_{L}B-L2-V_{H}B-L3-V_{L}A, V_{H}A-L1-V_{H}B-L2-V_{L}B-L3-V_{L}A, V_{L}A-L1-VLB-L2-V_{H}B-L3-V_{H}A, V_{L}A-L1-V_{H}B-L2-V_{L}B-L3-V_{H}A, V_{H}B-L1-V_{L}A-L2-V_{H}A-L3-V_{L}B, V_{H}B-L1-V_{H}A-L2-V_{L}A-L3-V_{L}B, V_{L}B-L1-V_{L}A-L2-V_{H}A-L3-V_{H}B or V_{L}B-L1-V_{H}A-L2-V_{L}A-L3-V_{H}B, particularly V_{L}B-L1-V_{H}A-L2-V_{L}A-L3-V_{H}B, wherein the V_{L}A and V_{H}A domains jointly form the antigen binding site for the first antigen, and V_{L}B and V_{H}B jointly form the antigen binding site for IL5R, particularly wherein linker L1 is a peptide of 2-10 amino acids, particularly 3-7 amino acids, particularly 5 amino acids, particularly GGGGS, linker L3 is a peptide of 1-10 amino acids, particularly 2-7 amino acids, particularly 5 amino acids, particularly GGGGS, and the linker L2 is a peptide of 10-40 amino acids, particularly 15 to 30 amino acids, particularly 20 to 25 amino acids, particularly 20 amino acids, particularly (GGGGS)₄.

In particular embodiments, said first antigen is CD3, and the V_{L}A and V_{H}A domains comprise the CDR regions SEQ ID NOs: 23 to 28, particularly the CDR regions SEQ ID NOs: 29 to 34 or the CDR regions SEQ ID NOs: 35 to 40, more particularly the CDR regions SEQ ID NOs: 41 to 46, the CDR regions SEQ ID NOs: 49 to 54, or the CDR regions SEQ ID NOs: 57 to 62, particularly wherein the V_{L}A framework regions are the framework regions selected from SEQ ID NO: 3 to 7, particularly SEQ ID NO: 3, and wherein the V_{H}A framework regions are the framework regions of SEQ ID NO: 8.

In particular embodiments, the V_{L}A domain comprise a sequence selected from SEQ ID NOs: 47, 55, and 63; and the V_{H}A domain comprise a sequence from SEQ ID NOs: 48, 56, and 64, particularly wherein the combination of a V_{L}A domain and a V_{H}A domain is selected from the combinations SEQ ID NO: 47 with SEQ ID NO: 48, SEQ ID NO: 55 with SEQ ID NO: 56, and SEQ ID NO: 63 with SEQ ID NO: 64.

In particular embodiments, said V_{L}B and V_{H}B domains comprise the CDR regions SEQ ID NOs: 9 to 14, particularly the CDR regions SEQ ID NOs: 15 to 20, particularly wherein the V_{L}B framework regions are the framework regions selected from SEQ ID NO: 3 to 7, particularly SEQ ID NO: 3, and wherein the V_{H}B framework regions are the framework regions of SEQ ID NO: 8.

In particular embodiments, the V_{L}B domain comprises SEQ ID NO: 21; and the V_{H}B domain comprises SEQ ID NO: 22.

In particular embodiments, the bispecific construct comprises: (i) a combination of a V_{L}A domain and a V_{H}A domain that is selected from the combinations: SEQ ID NO: 47 with SEQ ID NO: 48, SEQ ID NO: 55 with SEQ ID NO: 56, and SEQ ID NO: 63 with SEQ ID NO: 64, and (ii) the combination of a V_{L}B domain and a V_{H}B domain that is the combination of SEQ ID NO: 21 with SEQ ID NO: 22.

The bispecific constructs of the present invention may alternatively comprise one or more binding moieties based on non-antibody based binding domains. Specific examples of suitable methods for the preparation of the bispecific construct of the present invention further include, inter alia, the DARPin technology (Molecular Partners AG), the adnexin technology (Adnexus), the anticalin technology (Pieris), and the Fynomer technology (Covagen AG).

In a second aspect, the present invention relates to a nucleic acid or nucleic acids encoding the bispecific construct according to the present invention.

Thus, the present invention relates to a nucleic acid or multiple (i.e. more than one) nucleic acids encoding the bispecific construct of the present invention. If the bispecific construct is a single-chain construct, e.g. a polypeptide or protein, a single nucleic acid codes for the bispecific construct. However, if the bispecific construct comprises two or more polypeptides, the bispecific construct of the present invention may also be encoded by two or more separate nucleic acids. The nucleic acid molecule(s) according to the invention can be any nucleic acid molecule, particularly a DNA or RNA molecule, for example cDNA or mRNA. They can be naturally occurring molecules or produced through genetic engineering or chemical synthesis. They may be single-stranded molecules, which either contain the coding or the non-coding strand, or double-stranded molecules.

The nucleic acid(s) of the present invention may be produced by any suitable method as known to those skilled in the art. The nucleic acids of the present invention can, for example, be synthesized by the phosphoramidite method or the like, or can be produced by polymerase chain reaction (PCR) using specific primers. Furthermore, methods for introducing a desired mutation into certain nucleotide sequence, such as site-directed mutagenesis techniques, are well-known to a person skilled in the art.

In a third aspect, the present invention relates to a vector or vectors comprising the nucleic acid or nucleic acids according to the present invention.

Thus, the present invention relates to a vector or multiple vectors comprising the nucleic acid(s) of the present invention. When comprised within a vector, in particular a plasmid, the nucleic acid(s) particularly is (are) DNA. The types of vectors used in the present invention are not particularly limited. For example, the vector may be a vector which replicates autonomously, such as a plasmid, or may be a vector which is integrated into the genome of a host cell when introduced into the host cell and is replicated along with the chromosome. Particularly, the vector used in the present invention is an expression vector, in particular an expression plasmid. In an expression vector, elements necessary for transcription, such as a promoter, are operatively linked to the DNA nucleic acid(s) of the present invention.

Examples of promoters which are operative in bacterial cells include PR or PL promoters of phage lambda, lac, trp or tac promoter of Escherichia coli, and the like. Examples of mammalian promoters include SV40 promoter, MT-1 (metallothionein gene) promoter, adenovirus 2 major late promoter, and the like. Furthermore, exemplary promoters for use in insect cells include polyhedrin promoter, P10 promoter, baculovirus immediate early gene 1 promoter, and the like. Moreover, suitable promoters for yeast host cells include a promoter derived from yeast glycolysis system genes, TPI1 promoter and the like. Other promoters suited for different expression systems are known in the art.

Further, if necessary, the DNA of the present invention may be operatively linked to a suitable terminator, such as a human growth hormone terminator or a TPI1 ADH3 fungal host terminator. The recombinant vector of the present invention may also have an element such as a polyadenylation signal (e.g., derived from SV40), a transcription enhancer sequence (e.g. a SV40 enhancer), or a translation enhancer sequence (e.g., encoding adenovirus VA RNA).

The recombinant vector of the present invention is also typically provided with a DNA sequence which enables the vector to replicate inside the host cell, and an example thereof for mammalian cells is an SV40 origin of replication. Furthermore, the recombinant vector of the present invention may also contain a selectable marker. Examples of a selectable marker include, inter alia, drug resistance genes such as ampicillin, kanamycin, tetracycline, chloramphenicol, neomycin, and hygromycin. Methods for connecting the nucleic acid(s) of the present invention with a promoter and, as desired, other regulatory sequences such as a terminator and/or a secretion signal sequence, and inserting these into a suitable vector are known to those skilled in the art.

In a fourth aspect, the present invention relates to a host cell or host cells comprising the vector or vectors according to the present invention.

Thus, the present invention relates to a host cell or multiple host cells that are not identical, comprising the vector(s) of the present invention. The host cell(s) into which the recombinant vector of the present invention is (are) introduced is (are) not particularly limited and include any prokaryotic or eukaryotic cell which can express the vector of the present invention. Examples of suitable host cells include bacteria (e.g., Bacillus spp., Streptomyces spp., and Escherichia coli), mammalian cells (e.g., HEK293, HeLa, COS, BHK, CHL, and CHO cells), insect cells (e.g., baculovirus expression system), yeast cells (Saccharomyces spp. or Schizosaccharomyces spp., in particular Saccharomyces cerevisae and Saccharomyces kluyveri), and other fungal cells (e.g., Aspergillus, Neurospora). Particularly, the cells are bacterial cells, in particular Escherichia coli cells.

A multiple polypeptide chain bispecific construct can be made in a single host cell expression system wherein the host cell produces each chain of bispecific construct and assembles the polypeptide chains into a multimeric structure to form the bispecific construct, followed by recovery of the bispecific construct from the host cell. Alternatively, the separate polypeptide chains of the desired bispecific construct can be made in separate expression host cells, separately recovered from the respective host cells, and then mixed in vitro under conditions permitting the formation of the multi-subunit bispecific constructs as known in the art.

Methods for introducing the vector of the present invention into suitable host cells are known in the art and include the protoplast method, the competent cell method (for bacterial host cells), electroporation, the phosphate calcium method, lipofection (for mammalian cells or for insect cells/baculovirus system), electroporation, the spheroplast method, and the lithium acetate method (for yeast and other fungal host cells).

In a fifth aspect, the present invention relates to a method for producing the bispecific construct according to the present invention, comprising (i) providing a nucleic acid or nucleic acids according to the present invention, or a vector or vectors according to the present invention, expressing said nucleic acid or nucleic acids or said vector or vectors and collecting said bispecific construct from the expression system, or (ii) providing a host cell or host cells according to the present invention, culturing said host cell or said host cells; and collecting said bispecific construct from the cell culture.

Thus, the present invention relates to a method for producing the bispecific construct of the present invention, comprising providing a host cell or host cells of the present invention, culturing said host cell or said host cells and collecting the bispecific construct from the cell culture. In the culturing step, the host cell(s) of the present invention is (are) cultured in a suitable culture medium under conditions permitting expression of the bispecific construct of the present invention. The medium used to culture the cells may be any conventional medium suitable for growing the host cells, such as minimal or complex media containing appropriate supplements. Alternatively, the present invention relates to a method for producing the bispecific construct of the present invention, comprising providing a nucleic acid or nucleic acids according to the present invention, or a vector or vectors according to the present invention, expressing said nucleic acid or nucleic acids or said vector or vectors, particularly in an in vitro transcription/translation system (see, for example, Yin et al., MAbs 2012 Mar. 1; 4(2)), and collecting said bispecific construct from the expression system.

In the step of collecting the bispecific construct from the cell culture, the produced bispecific construct is recovered by conventional methods for isolating and purifying a protein, including separating the host cells from the medium by centrifugation or filtration, precipitating the proteinaceous components of the supernatant or filtrate by means of a salt, e.g. ammonium sulphate, and purification by a variety of chromatographic procedures, e.g. ion exchange chromatography, gel filtration chromatography, affinity chromatography or the like. In a case where the bispecific complex forms insoluble inclusion bodies, for example when using E. coli as host cell, the inclusion bodies may be first solubilized in denaturant, followed by a refolding step in accordance with procedures well known in the art.

In particular embodiments, the bispecific constructs of the present invention are expressed in E. coli. In particular embodiments, the bispecific constructs are obtained in functional form by refolding, particularly from inclusion bodies. In particular embodiments, the bispecific constructs are bispecific antibody-based constructs.

In a sixth aspect, the present invention relates to a pharmaceutical composition comprising the bispecific construct according to the present invention and a pharmaceutically acceptable carrier.

Thus, the present invention relates to a pharmaceutical composition comprising the bispecific construct of the present invention and a pharmaceutically acceptable carrier. As used herein, the term “pharmaceutically acceptable” refers to those compounds or substances which are, within the scope of sound medical judgment, suitable for contact with the tissues of mammals, especially humans, without excessive toxicity, irritation, allergic response and other problem complications. The term “carrier”, as used herein, relates to a diluent, adjuvant, excipient or vehicle whereby the active ingredient is administered. Pharmaceutically acceptable carriers for use herein can be, for example, sterile liquids or dispersions. Particular carriers are those suited for intravenous, subcutaneous or topical administration, including sterile aqueous and non-aqueous solutions or suspensions for parenteral administration, as discussed in Remington: The Science and Practice of Pharmacy, 20th Edition (2000).

The pharmaceutical composition generally includes an effective amount of the bispecific construct of the present invention. Within the present invention, the term “effective amount” refers to the amount of a compound sufficient to effect beneficial or desired therapeutic results. A therapeutically effective amount can be administered in one or more administrations, applications or dosages and is not intended to be limited to a particular formulation or administration route. Also, the pharmaceutical composition may include one or more additional active substances that are co-administered with the bispecific construct of the present invention. In addition, the pharmaceutical composition may contain additional pharmaceutically acceptable substances, for example pharmaceutical acceptable excipients such as solubilizing agents, surfactants, tonicity modifiers and the like.

Furthermore, the dosage form of the pharmaceutical composition of the present invention is not particularly limited but particularly is an inhalable formulation, such as an aqueous or non-aqueous solution or dispersion for nebulization, or any other formulation suited for topical administration. Another particular dosage form is a formulation containing the bispecific construct of the present invention formulated in a controlled release matrix. Further, the pharmaceutical composition may also be contained in an implantable device that releases the bispecific construct over time.

In a seventh aspect, the present invention relates to a bispecific construct according to the present invention, or the pharmaceutical composition according to the present invention, for use in the treatment of an indication in which eosinophils and/or basophils are critically involved in, particularly an allergic or inflammatory disease, particularly an allergic or inflammatory disease selected from asthma, atopic dermatitis, chronic obstructive pulmonary disease, eosinophilic gastrointestinal diseases, hyper-eosinophilic syndrome, Churg-Strauss syndrome, and eosinophilic nasal polyposis.

Thus, the present invention relates to the use of the bispecific construct of the present invention in the treatment of an allergic disease or other indication in which eosinophils and/or basophils play a critical role. In particular, the present invention relates to a method for the treatment of such diseases, comprising administering to a subject, particularly a human patient, an effective amount of the bispecific construct of the present invention. The meaning of the term “effective amount” is as defined herein above. Typically, an effective amount of the bispecific construct of the present invention is administered in form of the above-described pharmaceutical composition. Suitable administration routes include, but are not limited to, topical and parenteral administration, in particular inhalation, subcutaneous injection, intravenous injection, and injection into the cerebrospinal fluid. The administration regimen is not particularly limited and includes, for example, twice daily, daily, weekly, bi-weekly, monthly, once every other month, once every third, sixth or ninth month and once-a-year or single application administration schemes.

The allergic and inflammatory diseases may be asthma, atopic dermatitis, chronic obstructive pulmonary disease, eosinophilic gastrointestinal diseases, hyper-eosinophilic syndrome, Churg-Strauss syndrome, and eosinophilic nasal polyposis.

In a particular embodiment, said bispecific construct is for use in the topical application, particularly to the lung by inhalation. In particular such embodiments, the topical application is using a nebulizer, which generates aerosol droplets of a solution or suspension of a bispecific construct of the present invention, particularly a scDbs construct.

In particular embodiments, the bispecific construct has a remaining activity after nebulization of more than 90%, particularly more than 95%, more particularly of more than 98% of the activity prior to nebulization. Most particularly, the bispecific construct maintains its full activity after nebulization.

In particular embodiments, the bispecific construct has a loss of monomer content of less than 10%, particularly less than 5%, particularly less than 2%, and more particularly of less than 1% after nebulization. Most particularly, the bispecific construct has no loss of monomer content after nebulization (i.e. the bispecific construct maintains its monomeric form and does not form aggregates).

In particular embodiments, the bispecific construct shows degradation of less than 5%, particularly less than 2%, and more particularly of less than 1% after nebulization. Most particularly, the bispecific construct shows no degradation after nebulization.

In an eighth aspect the present invention relates to bispecific antibody constructs binding to CD3 and to IL5R that have a midpoint of thermal unfolding of at least 55° C., particularly more than 60° C., more particularly more than 65° C., and most particularly more than 70° C.

In particular embodiments, the bispecific antibody constructs comprise a sequence as disclosed in Sections [0064] to [0069] above.

In an ninth aspect the present invention relates to bispecific antibody fragments constructs binding to CD3 and to IL5R that show less than 20%, particularly less than 15%, more particularly less than 12%, even more particularly less than 10 and most particularly less than 5% loss in the monomer content of the single-chain diabody (scDb) when incubated at concentrations of 10 mg/ml in a simple saline buffer at 37° C. for 28 days.

In particular embodiments of such aspect, the bispecific antibody constructs comprise a sequence as disclosed in Sections [0064] to [0069] above.

In a tenth aspect, the present invention relates to a kit comprising (i) a bispecific construct or a pharmaceutical composition according to the present invention, and (ii) one or more of (x) a nebulizer; (y) a buffer or solvent for preparing a suspension or solution of the bispecific construct according to (i) to be nebulized; and/or (z) one or more ancillary reagents and/or tools for preparing a suspension or solution of the bispecific construct according to (i) to be nebulized.

### Examples

### Results

1. Binding of anti-CD3×anti-IL5R antibodies to Jurkat T cells and CHO-IL5R cells.

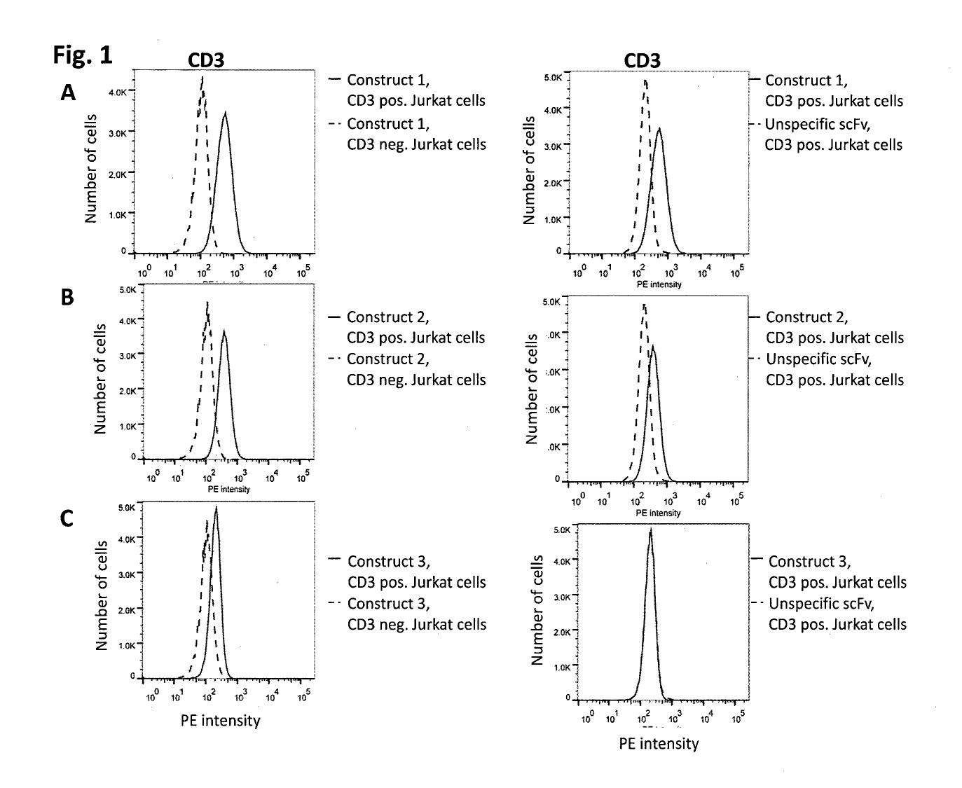

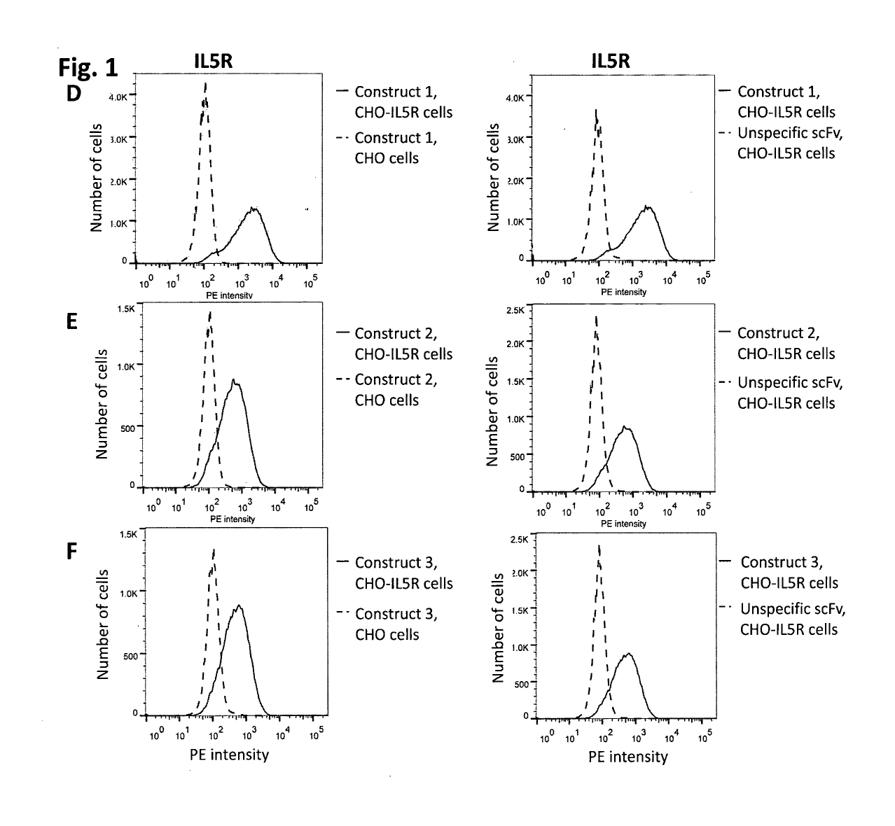

Jurkat T cells and IL5R-expressing CHO cells (CHO-IL5R) are incubated with 1 μg/ml and 10 μg/ml of the scDbs, as described in the methods section. With all scDbs tested, specific binding to CD3ε and IL5R expressing cell lines but no unspecific binding to control cell lines is detected. Three scDbs 1 to 3 closely related to the bispecific constructs disclosed in this application containing the identical anti-IL5R moiety while the anti-CD3 moieties being different, were tested. The anti-CD3 parts bind to overlapping epitopes with variable affinities (Table 1). As expected the binding to CHO-IL5R cells was similar for all scDbs tested (FIG. 1). In contrast binding to Jurkat T-cells decreased with decreasing affinity. No binding to Jurkat T-cells was detected for the low affinity binder Construct 3 at the highest concentration tested (FIG. 1).

2. Potential of Bispecific Anti-CD3×IL5R scDbs to Stimulate IL-2 Secretion from T Cells

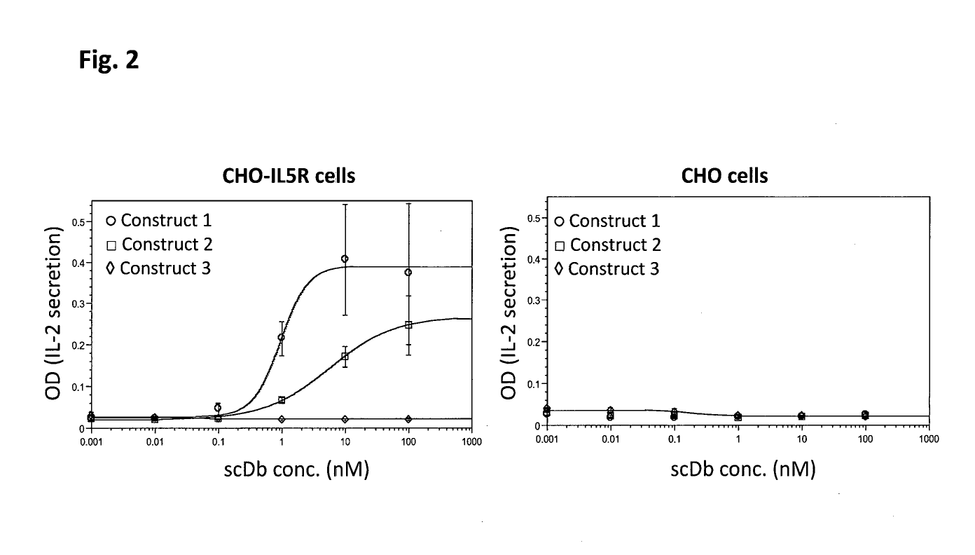

The potential of scDbs bound to a target cell to induce T-cell activation can be assessed by measurement of IL-2 secretion (see methods) by cytotoxic T-cells purified from human blood. The different scDbs are incubated with CD8+ cytotoxic T-cells in presence of target expressing CHO-IL5R cells at an effector:target cell ratio of 10:1 and IL-2 secretion is analysed after 16 hours of incubation. A dose-dependent stimulation of IL-2 secretion is observed in presence of CHO-IL5R cells while essentially no IL-2 secretion is observed in presence of wild-type CHO cells (see representative data for constructs 1 to 3 closely related to the constructs disclosed in this application in FIG. 2). Therefore, T-cell activation is only induced in presence of specific target cells. Moreover, the potential to induce IL-2 secretion correlates with binding affinity to recombinantly produced CD3εγ (Table 1) and to the capacity to bind to T-cells (FIG. 1). In line with affinity analysis, Construct 1, the binder with the highest affinity is a more potent inducer of IL-2 secretion than Construct 2, while no IL-2 secretion is observed with the low affinity scDb Construct 3 (FIG. 2).

3. Specific scDb Mediated Target Cell Lysis by Cytotoxic T-Cells

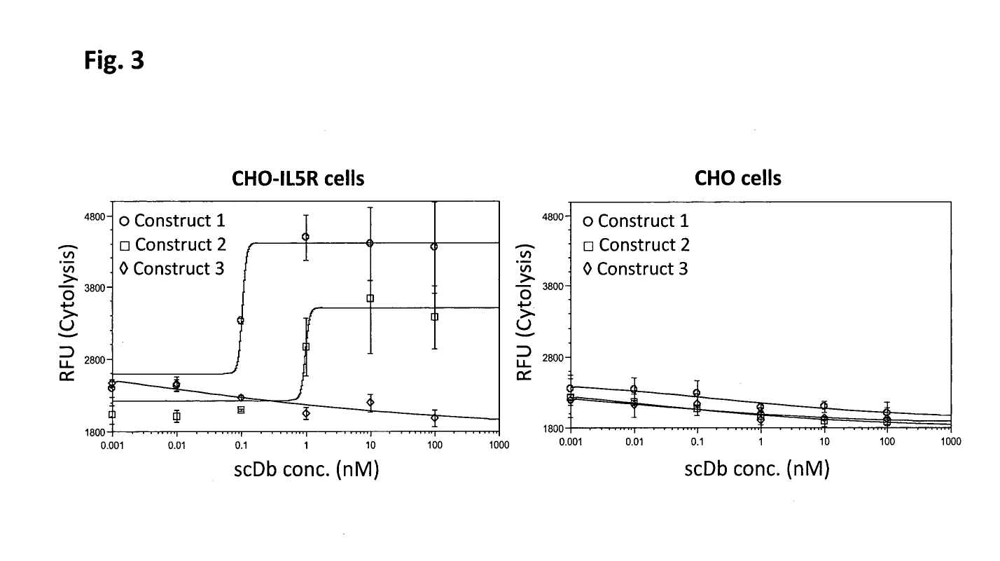

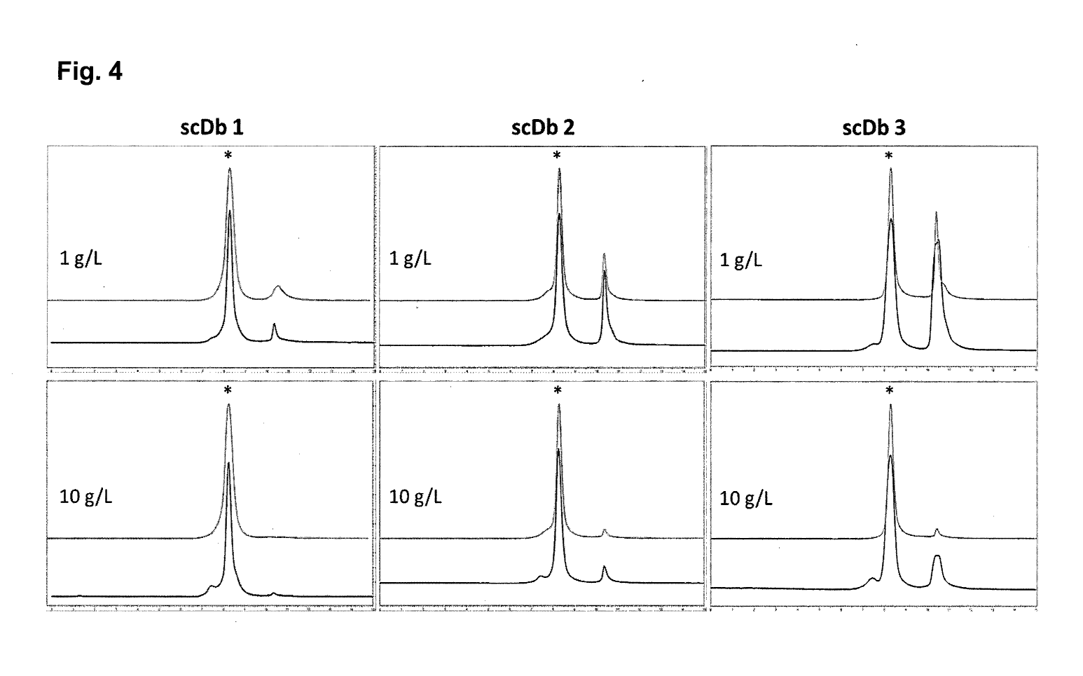

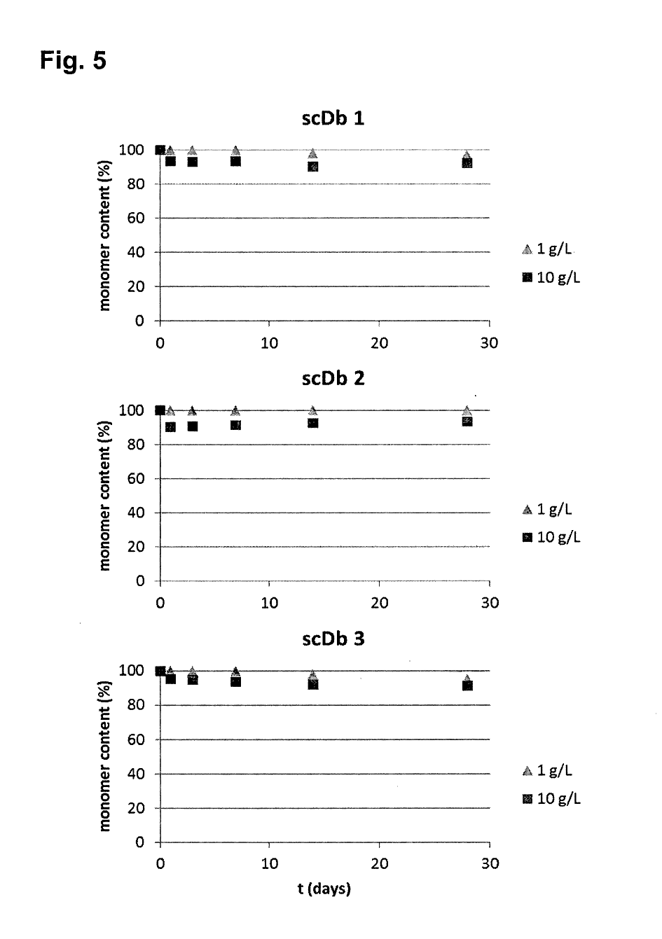

Specific lysis of target cells by cytotoxic T-cells mediated by anti-CD3×IL5R scDbs is analyzed with the CellTox™ green cytotoxicity assay (see methods) after 88 hours of incubation. Similarly to results discussed above for T-cell activation, a dose-dependent target cell lysis is observed for Construct 1 and Construct 2 in presence of CHO-IL5R cells while no lysis is observed in presence of wild-type CHO cells (see representative data for constructs 1 to 3 closely related to the constructs disclosed in this application in FIG. 3). In line with results mentioned above, scDbs binding with high affinity to CD3ε shows more potent lysis compared to the lower affinity scDbs. No target cell lysis is observed for the low affinity scDb Construct 3.

### Methods:

SPR Assay for Determination of Binding Kinetics of Bispecific Anti-CD3×IL5R scDbs

Binding affinities of anti-CD3×IL5R scDbs are measured by surface plasmon resonance (SPR) using a MASS-1 SPR instrument (Sierra Sensors). For affinity measurements to CD3, human heterodimeric single-chain CD3εγ extracellular domain (produced in-house) is immobilized on a sensor chip (SPR-2 Affinity Sensor High Capacity, Amine, Sierra Sensors) using a standard amine-coupling procedure. Three-fold serial dilutions of scDbs ranging from 90 to 0.1 nM are injected into the flow cells for 3 min and dissociation of the protein from the CD3εγ immobilized on the sensor chip is allowed to proceed for 12 min. After each injection cycle, surfaces are regenerated with two injections of 10 mM Glycine-HCl (pH 2.0). For affinity measurements against IL5R, an antibody specific for the Fc region of human IgGs is immobilized on a sensor chip (SPR-2 Affinity Sensor High Capacity, Amine, Sierra Sensors) by amine-coupling. A human IL5R-Fc chimeric protein (Novus Biologicals) is captured by the immobilized antibody. Three-fold serial dilutions of scDbs specific for IL5R (90 nM −0.1 nM) are injected into the flow cells for three minutes and dissociation is monitored for 12 minutes. After each injection cycle, surfaces are regenerated with three injections of 10 mM Glycine-HCl (pH 1.5). The apparent dissociation (kd) and association (ka) rate constants and the apparent dissociation equilibrium constant (K_{D}) are calculated with the MASS-1 analysis software (Analyzer, Sierra Sensors) using one-to-one Langmuir binding model.

Binding of Bispecific Anti-CD3×IL5R scDbs to CD3ε Expressed on the Cell Surface of T-Cells and to IL5R Expressed on the Surface of CHO Cells (CHO-IL5R Cells)

Binding of scDbs to CD3ε expressed on the cell surface of Jurkat cells (clone E6-1, ATCC), a human T cell line, is analyzed by flow cytometry. To assess unspecific binding of the scDbs to unknown components presented on the cell surface of Jurkat cells a CD3ε deficient derivative of the Jurkat T cell line (J.RT3-T3.5, ATCC) was used. Binding of scDbs to IL5R expressed on the cell-surface is analyzed using transgenic CHO-IL5R cells (generated at ZHAW) and wild-type CHO cells (Invitrogen) are used as controls for unspecific binding. Both cell lines are incubated with 1 μg/mL and 10 μg/mL of scDbs for 1 hour and bound scDbs are detected by addition of RPE-labeled protein L (BioVision) and then analyzed with a flow cytometer (FACS aria III, Becton Dickinson). As negative control a scFv specific for an unrelated target is used. For the qualitative assessment of binding to Jurkat and CHO-IL5R cells the mean fluorescence intensity (MFI), reflecting the signal intensity at the geometric mean, is measured for both, the unspecific scFv as well as for the test scDbs. The difference of the MFI between test antibody and negative control antibody (AMFI) is calculated as a measure for binding. Furthermore, the normalized MFI is calculated by dividing the MFI of the test scDb through the MFI of the negative control scFv.

T-Cell Activation by Bispecific Anti-CD3×IL5R scDbs: Induction of IL-2 Secretion

The potential of anti-CD3×anti-IL5R scDbs to induce IL-2 expression in CD8+ cytotoxic T-cells in presence of target cells is evaluated as follows. Cytotoxic T-cells are freshly isolated from human blood by using the RosetteSep™ human CD8+ T-cell enrichment cocktail (STEMCELL Technologies) according to the manufacturer's instructions. CHO-IL5R cells (10′000 cells/well) are incubated with CD8+ cytotoxic T-cells at an effector:target ratio of 10:1 in presence of 10-fold serially diluted scDbs (100 nM to 0.001 nM) in 96 well microtiter plates. To assess unspecific stimulation of T-cells wild-type CHO cells are used as target cells. Supernatant are collected after 16 hours of co-incubation to measure IL-2 release. IL-2 release is quantified using a commercially available ELISA kit (BioLegend). Data are analyzed using a four-parameter logistic curve fit using the SoftMax® Pro data analysis Software (Molecular Devices), and the molar concentration of scDb required to induce half maximal IL-2 secretion (EC₅₀) is derived from dose-response curves.

scDb Mediated Lysis of IL5R Expressing CHO Cells by Cytotoxic T Cells

For assessment of the potential of bispecific anti-CD3×IL5R scDbs to induce target cell lysis a transgenic IL5R expressing CHO cell line is used (CHO-IL5R). Unstimulated human CD8+ T-cells isolated as described above are used as effector cells. Target cells are labeled with cell tox green dye (Promega) according to the manufacturer's instructions. Cell lysis is monitored by the CellTox™ green cytotoxicity assay (Promega). The assay measures changes in membrane integrity that occur as a result of cell death. The assay uses an asymmetric cyanine dye that is excluded from viable cells but preferentially stains the dead cell DNA. When the dye binds DNA in compromised cells, its fluorescence properties are substantially enhanced. Viable cells produce no appreciable increases in fluorescence. Therefore, the fluorescence signal produced by the binding interaction with dead cell DNA is proportional to cytotoxicity. Similarly as described above, labeled CHO-IL5R cells (10′000 cells/well) are incubated with CD8+ cytotoxic T-cells at an effector:target ratio of 10:1 in presence of 10-fold serially diluted scDbs (100 nM to 0.001 nM) in 96 well microtiter plates. To assess unspecific lysis of cells that do not express the target, T-cells are co-incubated with labeled wild-type CHO cells. Fluorescence intensity is analyzed after 88 h of incubation using a multi-mode microplate reader (FlexStation 3, Molecular Devices). Data are analyzed using a four-parameter logistic curve fit using the SoftMax® Pro data analysis Software (Molecular Devices), and the molar concentration of scDb required to induce half maximal target cell lysis (EC₅₀) is derived from dose-response curves.

### Construct Design and Manufacture

The nucleotide sequences (see Tables 4 and 5) are de novo synthesized and cloned into an adapted vector for E. coli expression that is based on a pET26b(+) backbone (Novagen). The expression construct is transformed into the E. coli strain BL12 (DE3) (Novagen) and the cells are cultivated in 2YT medium (Sambrook, J., et al., Molecular Cloning: A Laboratory Manual) as a starting culture. Expression cultures are inoculated and incubated in shake flasks at 37° C. and 200 rpm. Once an OD600 nm of 1 is reached protein expression is induced by the addition of IPTG at a final concentration of 0.5 mM. After overnight expression the cells are harvested by centrifugation at 4000 g. For the preparation of inclusion bodies the cell pellet is resuspended in IB Resuspension Buffer (50 mM Tris-HCl pH 7.5, 100 mM NaCl, 5 mM EDTA, 0.5% Triton X-100). The cell slurry is supplemented with 1 mM DTT, 0.1 mg/mL Lysozyme, 10 mM Leupeptin, 100 μM PMSF and 1 μM Pepstatin. Cells are lysed by 3 cycles of ultrasonic homogenization while being cooled on ice. Subsequently 0.01 mg/mL DNAse is added and the homogenate is incubated at room temperature for 20 min. The inclusion bodies are sedimented by centrifugation at 15000 g and 4° C. The IBs are resuspended in IB Resuspension Buffer and homogenized by sonication before another centrifugation. In total a minimum of 3 washing steps with IB Resuspension Buffer are performed and subsequently 2 washes with IB Wash Buffer (50 mM Tris-HCl pH 7.5, 100 mM NaCl, 5 mM EDTA) are performed to yield the final IBs.

For protein refolding the isolated IBs are resuspended in Solubilization Buffer (100 mM Tris/HCl pH 8.0, 6 M Gdn-HCl, 2 mM EDTA) in a ratio of 5 mL per g of wet IBs. The solubilization is incubated for 30 min at room temperature until DTT is added at a final concentration of 20 mM and the incubation is continued for another 30 min. After the solubilization is completed the solution is cleared by 10 min centrifugation at 21500 g and 4° C. The refolding is performed by rapid dilution at a final protein concentration of 0.3 g/L of the solubilized protein in Refolding Buffer (typically: 100 mM Tris-HCl pH 8.0, 5.0 M Urea, 5 mM Cysteine, 1 mM Cystine). The refolding reaction is routinely incubated for a minimum of 14 h. The resulting protein solution is cleared by 10 min centrifugation at 8500 g and 4° C. The refolded protein is purified by affinity chromatography on Capto L resin (GE Healthcare). The isolated monomer fraction is analyzed by size-exclusion HPLC, SDS-PAGE for purity and UV/Vis spectroscopy for protein content. Buffer is exchange into Native buffer (50 mM Citrate-Phosphate pH 6.4, 200 mM NaCl) by dialysis. The protein concentration is adjusted to the intended value and the stability analysis is performed.

### Thermal Unfolding

The midpoint of transition for the thermal unfolding of the tested constructs is determined by Differential Scanning Fluorimetry (DSF), essentially as described by Niesen (Niesen et al., Nat Protoc. 2 (2007) 2212-21). The DSF assay is performed in a qPCR machine (e.g. MX3005p, Agilent Technologies). The samples are diluted in buffer (citrate-phosphate pH 6.4, 0.25 M NaCl) containing a final concentration of 5× SYPRO orange in a total volume of 25 μL. Samples are measured in triplicates and a temperature ramp from 25-96° C. programmed. The fluorescence signal is acquired and the raw data is analyzed with the GraphPad Prism (GraphPad Software Inc.). Representative data created using constructs closely related to those disclosed in this application are shown in Table 2.

### Stress Stability Study

The protein is analyzed over the course of four weeks and storage at 37° C. with respect to oligomerization by size-exclusion high-performance liquid chromatography (SE-HPLC) and degradation by sodium dodecyl sulfate polyacrylamide gel electrophoresis (SDS-PAGE). Prior to the study the samples are concentrated to 1 and 10 g/L and starting time points are determined. The monomer content is quantified by separation of the samples on a Shodex KW-402.5-4F (Showa Denko) and evaluation of the resulting chromatograms. For the calculation of the relative percentage of protein monomer the area of the monomeric peak is divided by the total area of peaks that cannot be attributed to the sample matrix. The protein degradation is assessed by SDS-PAGE analysis with Any kD Mini-Protean TGX gels (Bio-Rad Laboratories) and stained with Coomassie brilliant blue. The protein concentration is monitored at the different time points by UV-Vis spectroscopy with an Infinity reader M200 Pro equipped with a Nanoquant plate (Tecan Group Ltd.). Representative data created using constructs closely related to those disclosed in this application are shown in Table 3.

### Stability During Nebulization

The scDbs formulated in an aqueous solution at increasing concentrations ranging from 0.01 to 10 mg/ml are subjected to nebulization/aerosolization using a medical nebulizer based on a principle such as the vibrating mesh technology, jet nebulization or ultrasonic wave nebulization. Aerosols harvested from the mouth piece and formulation residuals collected from the liquid reservoir of the device are subjected to further analysis to confirm their integrity and activity. SPR and ELISA are used to measure the activity of the samples, SE-HPLC is used to determine the loss of monomer content during nebulization, and degradation is assessed by SDS-Page.

TABLE 1
Pharmacodynamic and biophysical characteristics of anti-CD3 × anti-IL5R scDbs closely related to those disclosed in
this application. For the qualitative detection of binding of scDbs to target cells the mean fluorescence intensity (MFI),
reflecting the signal intensity at the geometric mean, was measured for both, the negative control as well as for the test
scDbs. The normalized MFI was calculated by dividing the MFI of the test scDb through the MFI of the negative control scFv.
Anti-IL5R × CD3E single-chain diabodies

| | | | | | | | | | | Potency | | | |
|---|---|---|---|---|---|---|---|---|---|---|---|---|---|
| | | | | | | | | Binding | Potency | to | | % monomer | |
| | | | | | | | Binding | to | to lyse | induce | | loss | |
| | | | | | | | to | CHO- | target | IL-2 | | (4 w at | |
| | SPR Data human IL-5Ra | | | SPR Data human CD3γε | | | Jurkat | IL5Ra | cells | secretion | | 37° C.) | |
| scDb | ka | kd | KD | ka | kd | KD | cells | cells | EC₅₀ | EC₅₀ | Tm | 1 | 10 |
| ID | [M⁻¹ s⁻¹] | [s⁻¹] | [M] | [M⁻¹ s⁻¹] | [s⁻¹] | [M] | nMFI* | nMFI* | [nM] | [nM] | [° C.] | g/L | g/L |
| Con- | 1.14E+06 | 3.99E−05 | 3.50E−11 | 1.85E+05 | 2.12E−03 | 1.15E−08 | 2.9 | 18.8 | 0.10 | 0.96 | 67.90 ± 0.05 | 3.17 | 7.87 |
| struct | | | | | | | | | | | | | |
| 1 | | | | | | | | | | | | | |
| Con- | 1.89E+06 | 5.57E−05 | 2.95E−11 | 1.11E+05 | 3.27E−03 | 2.96E−08 | 1.9 | 16.7⁺ | 0.96 | 5.67 | 71.93 ± 0.09 | 0 | 6.63 |
| struct | | | | | | | | | | | | | |
| 2 | | | | | | | | | | | | | |
| Con- | 1.66E+06 | <1E−06 | <6.04E−13 | 1.99E+05 | 2.45E−02 | 1.23E−07 | 1.0 | 15.6⁺ | no | no IL-2 | 69.11 ± 0.02 | 5.54 | 11.07 |
| struct | | | | | | | | | lysis | secretion | | | |
| 3 | | | | | | | | | | | | | |

*MFI normalized to negative ctrl.
⁺multiplied by correction factor 3.1 (new lot of protein L)

TABLE 2
The midpoint of transition for the thermal unfolding was determined
for constructs closely related to those disclosed in this
application by differential scanning fluorimetry

| Construct ID | Tm |
|---|---|
| 1 | 67.90 ± 0.05 |
| 2 | 71.93 ± 0.09 |
| 3 | 69.11 ± 0.02 |

TABLE 3
Monomer loss for constructs closely related to those disclosed in this
application during stress stability over the duration of 28 days

| SEQ ID | 1 g/L at 37° C. | 10 g/L at 37° C. |
|---|---|---|
| 1 | 3.17 | 7.87 |
| 2 | 0 | 6.63 |
| 3 | 5.54 | 11.07 |

### Sequences

TABLE 4
List of protein sequences

| SEQ | | |
|---|---|---|
| ID | Type | Sequence |
| 1 | Linker long | GGGGSGGGGSGGGGSGGGGS |
| 2 | Linker short | GGGGS |
| 3 | Sk6 | DIQMTQSPSSLSASVGDRVTITC (CDR-L1) |
| | | WYQQKPGKAPKLLIY (CDR-L2) |
| | | GVPSRFSGSGSGTDFTLTISSLQPEDFATYYC (CDR-L3) |
| | | FGQGTKLTVLG |
| 4 | Sk12 | DIQMTQSPSSLSASVGDRVTITC (CDR-L1) |
| | | WYQQKPGKAPKLLIY (CDR-L2) |
| | | GVPSRFSGSGSGTDFTLTISSLQPEDFATYYC (CDR-L3) |
| | | FGGGTKLTVLG |
| 5 | Sk16 | DIQMTQSPSSLSASVGDRVTITC (CDR-L1) |
| | | WYQQKPGKAPKLLIY(CDR-L2) |
| | | GVPSRFSGSGSGTDFTLTISSLQPEDEATYYC (CDR-L3) |
| | | FGGGTKLTVLG |
| 6 | Sk17 | DIQMTQSPSSLSASVGDRVTITC (CDR-L1) |
| | | WYQQKPGKAPKLLIY (CDR-L2) |
| | | GVPSRFSGSGSGTDFTLTISSLQPEDFATYYC (CDR-L3) |
| | | FGTGTKVTVLG |
| 7 | Sk18 | DIQMTQSPSSLSASVGDRVTITC (CDR-L1) |
| | | WYQQKPGKAPKLLIY (CDR-L2) |
| | | GVPSRFSGSGSGTDFTLTISSLQPEDEATYYC (CDR-L3) |
| | | FGTGTKVTVLG |
| 8 | Sh4 | EVQLVESGGGLVQPGGSLRLSCAAS (CDR-H1) |
| | | WVRQAPGKGLEWIG (CDR-H2) |
| | | RFTISRDNSKNTVYLQMNSLRAEDTAVYYCA (CDR-H3) |
| | | WGQGTLVTVSS |
| 9 | Cluster 1 | QASX(Q/E)NIYSNLA |
| | CDR L1 | |
| 10 | Cluster 1 | RASTLAS |
| | CDR L2 | |
| 11 | Cluster 1 | QSNYGINX(Y/I)YGAA |
| | CDR L3 | |
| 12 | Cluster 1 | GFSLSSYDMX(T/S) |
| | CDR H1 | |
| 13 | Cluster 1 | IIYX(V/T)SX(G/A)SX(A/T)YYASWAKG |
| | CDR H2 | |
| 14 | Cluster 1 | RX(I/V)NYGX(L/M)DL |
| | CDR H3 | |
| 15 | 03-19-C08 | QASQNIYSNLA |
| | CDR L1 | |
| 16 | 03-19-C08 | RASTLAS |
| | CDR L2 | |
| 17 | 03-19-C08 | QSNYGINYYGAA |
| | CDR L3 | |
| 18 | 03-19-C08 | GFSLSSYDMT |
| | CDR H1 | |
| 19 | 03-19-C08 | IIYVSGSAYYASWAKG |
| | CDR H2 | |
| 20 | 03-19-C08 | RINYGLDL |
| | CDR H3 | |
| 21 | 03-19-C08 | DIQMTQSPSSLSASVGDRVTITCQASQNIYSNLAWYQQKP |
| | sc03 Sk6 | GKAPKLLIYRASTLASGVPSRFSGSGSGTDFTLTISSLQPED |
| | | FATYYCQSNYGINYYGAAFGQGTKLTVLG |
| 22 | 03-19-C08 | EVQLVESGGGLVQPGGSLRLSCAASGFSLSSYDMTWVRQ |
| | sc03 Sh4 | APGKGLEWIGIIYVSGSAYYASWAKGRFTISRDNSKNTVYL |
| | | QMNSLRAEDTAVYYCARINYGLDLWGQGTLVTVSS |
| 23 | Cluster 2 | QSX(S/N)X(E/Q)X(S/N)X(V/I)YX(S/N)NX(N/K)RLS |
| | CDR-L1 | |
| 24 | Cluster 2 | X(S/T)X(A/T)SX(S/T)LAS |
| | CDR-L2 | |
| 25 | Cluster 2 | QGEFX(S/N/T)CX(S/N)X(S/N/R)X(A/V)DCFX(T/S/N) |
| | CDR-L3 | |
| 26 | Cluster 2 | GFPLX(S/N)X(S/A/R)X(Y/F)AMX(L/I/G) |
| | CDR-H1 | |
| 27 | Cluster 2 | X(M/L)IX(L/T/M/I)RX(A/S)X(D/G/H)X(N/K/T)X |
| | CDR-H2 | (K/T/I/M/V)YYAX(S/N)WX(A/V)X(K/N)G |
| 28 | Cluster 2 | RRX(H/Q)YNX(T/A/R)X(D/Y/S/E)GX(Y/N)PX(I/V)X |
| | CDR-H3 | (G/A)IGDL |
| 29 | Cluster 2.1 | QSSX(E/Q)SVYX(S/N)NX(N/K)RLS |
| | CDR-L1 | |
| 30 | Cluster 2.1 | X(S/T)X(A/T)SX(S/T)LAS |
| | CDR-L2 | |
| 31 | Cluster 2.1 | QGEFX(S/T)CSX(S/N/R)X(A/V)DCFX(T/S/N) |
| | CDR-L3 | |
| 32 | Cluster 2.1 | GFPLSX(S/A)YAMX(I/G) |
| | CDR-H1 | |
| 33 | Cluster 2.1 | MIX(L/I)RX(A/S)X(G/D)X(N/T)X(T/I/V)YYAX(S/N)WX |
| | CDR-H2 | (A/V)X(K/N)G |
| 34 | Cluster 2.1 | RRX(H/Q)YNX(T/A/R)X(D/Y/S/E)GX(Y/N)PX(I/V)X |
| | CDR-H3 | (G/A)IGDL |
| 35 | Cluster 2.2 | QSSESVYNNX(N/K)RLS |
| | CDR-L1 | |
| 36 | Cluster 2.2 | X(S/T)ASSLAS |
| | CDR-L2 | |
| 37 | Cluster 2.2 | QGEFX(S/T)CSX(S/N)ADCFT |
| | CDR-L3 | |
| 38 | Cluster 2.2 | GFPLSSYAMI |
| | CDR-H1 | |
| 39 | Cluster 2.2 | MILRAGNIYYASWX(A/V)KG |
| | CDR-H2 | |
| 40 | Cluster 2.2 | RRX(H/Q)YNX(T/R)X(D/E)GYPIGIGDL |
| | CDR-H3 | |
| 41 | 09-24-H09 | QSSESVYNNKRLS |
| | CDR L1 | |
| 42 | 09-24-H09 | TASSLAS |
| | CDR L2 | |
| 43 | 09-24-H09 | QGEFTCSNADCFT |
| | CDR L3 | |
| 44 | 09-24-H09 | GFPLSSYAMI |
| | CDR H1 | |
| 45 | 09-24-H09 | MILRAGNIYYASINVKG |
| | CDR H2 | |
| 46 | 09-24-H09 | RRHYNREGYPIGIGDL |
| | CDR-H3 | |
| 47 | 09-24-H09 | DIQMTQSPSSLSASVGDRVTITCQSSESVYNNKRLSWYQQ |
| | sc01 Sk6 | KPGKAPKLLIYTASSLASGVPSRFSGSGSGTDFTLTISSLQP |
| | | EDFATYYCQGEFTCSNADCFTFGQGTKLTVLG |
| 48 | 09-24-H09 | EVQLVESGGGLVQPGGSLRLSCAASGFPLSSYAMIWVRQA |
| | sc01 Sh4 | PGKGLEWIGMILRAGNIYYASWVKGRFTISRDNSKNTVYLQ |
| | | MNSLRAEDTAVYYCARRHYNREGYPIGIGDLWGQGTLVTV |
| | | SS |
| 49 | 09-19-F11 | QSSESVYSNNRLS |
| | CDR L1 | |
| 50 | 09-19-F11 | SASTLAS |
| | CDR L2 | |
| 51 | 09-19-F11 | QGEFSCSSVDCFS |
| | CDR L3 | |
| 52 | 09-19-F11 | GFPLSAYAMI |
| | CDR H1 | |
| 53 | 09-19-F11 | MIIRSGTVYYANWAKG |
| | CDR H2 | |
| 54 | 09-19-F11 | RRHYNADGYPIGIGDL |
| | CDR H3 | |
| 55 | 09-19-F11 | DIQMTQSPSSLSASVGDRVTITCQSSESVYSNNRLSWYQQ |
| | sc01 Sk6 | KPGKAPKLLIYSASTLASGVPSRFSGSGSGTDFTLTISSLQP |
| | | EDFATYYCQGEFSCSSVDCFSFGQGTKLTVLG |
| 56 | 09-19-F11 | EVQLVESGGGLVQPGGSLRLSCAASGFPLSAYAMIWVRQA |
| | sc01 Sh4 | PGKGLEWIGMIIRSGTVYYANWAKGRFTISRDNSKNTVYLQ |
| | | MNSLRAEDTAVYYCARRHYNADGYPIGIGDLWGQGTLVTV |
| | | SS |
| 57 | 09-23-C07 | QSNENIYSNNRLS |
| | CDR L1 | |
| 58 | 09-23-C07 | SASSLAS |
| | CDR L2 | |
| 59 | 09-23-C07 | QGEFNCNSADCFT |
| | CDR L3 | |
| 60 | 09-23-C07 | GFPLNRYAML |
| | CDR H1 | |
| 61 | 09-23-C07 | LITRADKKYYASWAKG |
| | CDR H2 | |
| 62 | 09-23-C07 | RRHYNTDGYPIAIGDL |
| | CDR H3 | |
| 63 | 09-23-C07 | DIQMTQSPSSLSASVGDRVTISCQSNENIYSNNRLSWYQQ |
| | sc02 Sk6 | KPGKAPKLLIYSASSLASGVPSRFSGSGSGTDFTLTISDLEP |
| | | EDFATYYCQGEFNCNSADCFTFGQGTKLTVLG |
| 64 | 09-23-C07 | EVQLVESGGGLVQPGGSLRLSCAVSGFPLNRYAMLWVRQ |
| | sc02 Sh4 | APGKGLEWIGLITRADKKYYASWAKGRFTISKDNSKNTVYL |
| | | QMNSLRAEDTAVYYCARRHYNTDGYPIAIGDLWGQGTLVT |
| | | VSS |

Note:
″X(....)″ indicates a position with sequence variability, wherein the amino acid residues permitted at the given position are shown in parenthesis
Example:
″X(A/C)″ indicates a position in the protein sequence, where the two amino acids A and C are permitted

TABLE 5
Combinations of variable domains for the different scFv constructs

| scDb | VL 1 | Linker | VH 2 | Linker | VL 2 | Linker | VH1 |
|---|---|---|---|---|---|---|---|
| construct | [SEQ ID] | [SEQ ID] | [SEQ ID] | [SEQ ID] | [SEQ ID] | [SEQ ID] | [SEQ ID] |
| 1 | 21 | 2 | 48 | 1 | 47 | 2 | 22 |
| 2 | 21 | 2 | 56 | 1 | 55 | 2 | 22 |
| 3 | 21 | 2 | 64 | 1 | 63 | 2 | 22 |

We claim:

1. A bispecific construct comprising at least one first binding moiety and at least one second binding moiety, wherein said first binding moiety specifically binds to a first antigen present on a cytotoxic effector T (Tc) cell, and said second binding moiety specifically binds to the IL-5 receptor (IL5R) present on the surface of a target cell, particularly wherein the target cell is an eosinophil or basophil cell, particularly wherein the second binding moiety has an equilibrium dissociation constant K_{D} of 10⁻¹⁰ M or less, particularly of less than 5×10⁻¹¹ M.

2. The bispecific construct of claim 1, wherein said first binding moiety specifically binds to an antigen selected from CD3 and CD28.

3. The bispecific construct of claim 1, wherein said first binding moiety specifically binds to CD3, particularly to the epsilon chain of CD3 (CD3E), more particularly to an agonistic epitope of CD3E.

4. The bispecific construct of claim 1, wherein the construct allows for efficient killing of said target cell by the Tc cell and/or wherein said Tc cell is a stimulated or an unstimulated Tc cell.

5. The bispecific construct of claim 1, wherein said first binding moiety and/or said second binding moiety is an antibody-based binding moiety, particularly an antibody-based binding moiety comprising a heavy chain variable domain (V_{H}) or binding fragment thereof, more particularly an antibody-based binding moiety comprising a heavy chain variable domain (V_{H}) or binding fragment thereof, and a light chain variable domain (V_{L}) or binding fragment thereof.

6. The bispecific construct of claim 5, which is an antibody format selected from the group consisting of a single-chain diabody (scDb), a tandem scDb (Tandab), a linear dimeric scDb (LD-scDb), a circular dimeric scDb (CD-scDb), a bispecific T-cell engager (BiTE; tandem di-scFv), a disulfide-stabilized Fv fragment, a tandem tri-scFv, a tri(a)body, bispecific Fab₂, di-miniantibody, tetrabody, scFv-Fc-scFv fusion, di-diabody, DVD-Ig, IgG-scFab, scFab-dsscFv, Fv2-Fc, IgG-scFv fusions, including bsAb, Bs1Ab, Bs2Ab, Bs3Ab, Ts1Ab, Ts2Ab, and Knob-into-Holes (KiHs) and DuoBodies, an VH and a VL domain, each fused to the C-terminus of the two different heavy chains of a KiHs or DuoBody such that a functional Fv domain is formed, particularly a single-chain diabody (scDb), and more particularly a bispecific monomeric scDb.

7. The bispecific construct of claim 6, wherein the bispecific scDb comprises two variable heavy chain domains (V_{H}) or fragments thereof and two variable light chain domains (V_{L}) or fragments thereof connected by linkers L1, L2 and L3 in the order V_{H}A-L1-V_{L}B-L2-V_{H}B-L3-V_{L}A, V_{H}A-L1-V_{H}B-L2-V_{L}B-L3-V_{L}A, V_{L}A-L1-V_{L}B-L2-V_{H}B-L3-V_{H}A, V_{L}A-L1-V_{H}B-L2-V_{L}B-L3-V_{H}A, V_{H}B-L1-V_{L}A-L2-V_{H}A-L3-V_{L}B, V_{H}B-L1-V_{H}A-L2-V_{L}A-L3-V_{L}B, V_{L}B-L1-V_{L}A-L2-V_{H}A-L3-V_{H}B or V_{L}B-L1-V_{H}A-L2-V_{L}A-L3-V_{H}B, particularly V_{L}B-L1-V_{H}A-L2-V_{L}A-L3-V_{H}B, wherein the V_{L}A and V_{H}A domains jointly form the antigen binding site for the first antigen, and V_{L}B and V_{H}B jointly form the antigen binding site for IL5R, particularly wherein linker L1 is a peptide of 2-10 amino acids, particularly 5 amino acids, particularly GGGGS, linker L3 is a peptide of 1-10 amino acids, particularly 5 amino acids, particularly GGGGS, and the linker L2 is a peptide of 10-40 amino acids, particularly 20 amino acids, particularly (GGGGS)₄.

8. The bispecific construct of claim 1, wherein the first antigen is CD3, and wherein the V_{L}A and V_{H}A domains comprise the CDR regions SEQ ID NOs: 23 to 28, particularly the CDR regions SEQ ID NOs: 29 to 34 or the CDR regions SEQ ID NOs: 35 to 40, more particularly the CDR regions SEQ ID NOs: 41 to 46, the CDR regions SEQ ID NOs: 49 to 54, or the CDR regions SEQ ID NOs: 57 to 62, particularly wherein the V_{L}A framework regions are the framework regions selected from SEQ ID NO: 3 to 7, particularly SEQ ID NO: 3, and wherein the V_{H}A framework regions are the framework regions of SEQ ID NO: 8.

9. The bispecific construct of claim 6, wherein the V_{L}A domain comprise a sequence selected from SEQ ID NOs: 47, 55, and 63; and the V_{H}A domain comprise a sequence from SEQ ID NOs: 48, 56, and 64, particularly wherein the combination of a V_{L}A domain and a V_{H}A domain is selected from the combinations SEQ ID NO: 47 with SEQ ID NO: 48, SEQ ID NO: 55 with SEQ ID NO: 56, and SEQ ID NO: 63 with SEQ ID NO: 64.

10. The bispecific construct of claim 1, wherein the V_{L}B and V_{H}B domains comprise the CDR regions SEQ ID NOs: 9 to 14, particularly the CDR regions SEQ ID NOs: 15 to 20, particularly wherein the V_{L}B framework regions are the framework regions selected from SEQ ID NO: 3 to 7, particularly SEQ ID NO: 3, and wherein the V_{H}B framework regions are the framework regions of SEQ ID NO: 8.

11. The bispecific construct of claim 10, wherein the V_{L}B domain comprises SEQ ID NO: 21; and the V_{H}B domain comprises SEQ ID NO: 22.

12. The bispecific construct of claim 7, wherein the combination of a V_{L}A domain and a V_{H}A domain is selected from the combinations SEQ ID NO: 47 with SEQ ID NO: 48, SEQ ID NO: 55 with SEQ ID NO: 56, and SEQ ID NO: 63 with SEQ ID NO: 64, and wherein the combination of a V_{L}B domain and a V_{H}B domain is the combination SEQ ID NO: 21 with SEQ ID NO: 22.

13. A nucleic acid or nucleic acids encoding the bispecific construct according to claim 1.

14. A vector or vectors comprising the nucleic acid or nucleic acids according to claim 13.

15. A host cell or host cells comprising the vector or vectors according to claim 14.

16. A method for producing a bispecific construct, comprising providing a nucleic acid or nucleic acids of claim 13, expressing said nucleic acid or nucleic acids and collecting said bispecific construct from the expression system.

17. A pharmaceutical composition comprising the bispecific construct of claim 1 and a pharmaceutically acceptable carrier.

18. A bispecific construct of claim 1, for use in the treatment of an indication in which eosinophils and/or basophils are critically involved in, particularly an allergic or inflammatory disease, particularly an allergic or inflammatory disease selected from asthma, atopic dermatitis, chronic obstructive pulmonary disease, eosinophilic gastrointestinal diseases, hyper-eosinophilic syndrome, Churg-Strauss syndrome, and eosinophilic nasal polyposis.

19. A method for the treatment of an indication in which eosinophils and/or basophils are critically involved in, particularly an allergic or inflammatory disease, particularly an allergic or inflammatory disease selected from asthma, atopic dermatitis, chronic obstructive pulmonary disease, eosinophilic gastrointestinal diseases, hyper-eosinophilic syndrome, Churg-Strauss syndrome, and eosinophilic nasal polyposis, comprising the step of administering the bispecific construct of claim 1.

20. A bispecific antibody fragment binding to CD3 and to IL5R that has a midpoint of thermal unfolding of at least 55° C., particularly more than 60° C., more particularly more than 65° C., and most particularly more than 70° C.

21. A bispecific antibody fragment binding to CD3 and to IL5R that shows less than 20%, particularly less than 15%, more particularly less than 12%, even more particularly less than 10 and most particularly less than 5% loss in the monomer content of the single-chain diabody (scDb) when incubated at concentrations of 10 mg/ml in a saline buffer at 37° C. for 28 days.

22. A kit comprising (i) the bispecific construct of claim 1, and (ii) one or more of (x) a nebulizer; (y) a buffer or solvent for preparing a suspension or solution of said bispecific construct to be nebulized; and/or (z) one or more ancillary reagents and/or tools for preparing a suspension or solution of said bispecific construct to be nebulized.
